SEQUENCE LISTING (152 sequences)

SEQ ID NO: 1 - protein, 9 aa, Artificial
  Synthetic
XYXWXERQL

SEQ ID NO: 2 - protein, 10 aa, Artificial
  Synthetic
XYXWXERQLG

SEQ ID NO: 3 - protein, 10 aa, Artificial
  Synthetic
XYGWXERQXG

SEQ ID NO: 4 - protein, 10 aa, Artificial
  Synthetic
XYXWXERQLG

SEQ ID NO: 5 - protein, 12 aa, Artificial
  Synthetic
SEGFYNAIELLS

SEQ ID NO: 6 - protein, 18 aa, Artificial
  Synthetic
XLEXEWXXXXXXVYXXXX

SEQ ID NO: 7 - protein, 18 aa, Artificial
  Synthetic
SLEEEWAQIEXEVWGRGX

SEQ ID NO: 8 - protein, 32 aa, Artificial
  Synthetic
SLEEEWAQIEAEVWGRGAPSESFYDWFERQLG

SEQ ID NO: 9 - protein, 34 aa, Artificial
  Synthetic
SLEEEWAQIEAEAEVWGRGAPSESFYDWFERQLG

SEQ ID NO: 10 - protein, 18 aa, Artificial
  Synthetic
XLEXEWXXXXXXVYXXXX

SEQ ID NO: 11 - protein, 18 aa, Artificial
  Synthetic
SLEEEWAQIXXEVWGRGX

SEQ ID NO: 12 - protein, 18 aa, Artificial
  Synthetic
XLEXEWXXXXXXVYXXXX

SEQ ID NO: 13 - protein, 32 aa, Artificial
  Synthetic
HQLEEEWQAIQCELWGRGCPSESFYDWFERQL

SEQ ID NO: 14 - protein, 31 aa, Artificial
  Synthetic
HLEEEWSEIQCELWGRGCPSESFYDWFERQL

SEQ ID NO: 15 - protein, 34 aa, Artificial
  Synthetic
HQLEEEWQAIQCELWGRGCPSEDFYDWFEAQLHA

SEQ ID NO: 16 - protein, 33 aa, Artificial
  Synthetic
HLEEEWSEIQCELWGRGCPSEDFYDWFEAQLHA

SEQ ID NO: 17 - protein, 34 aa, Artificial
  Synthetic
HELEEEWKRIECELWGRGCPSEDFYDWFEAQLHA

SEQ ID NO: 18 - protein, 34 aa, Artificial; identical to SEQ ID NO: 15
  Synthetic

SEQ ID NO: 19 - protein, 33 aa, Artificial; identical to SEQ ID NO: 16
  Synthetic

SEQ ID NO: 20 - protein, 34 aa, Artificial; identical to SEQ ID NO: 17
  Synthetic

SEQ ID NO: 21 - protein, 42 aa, Artificial
  Synthetic
SLEEEWAQIECEVYGRGCPSVRGFQGGTVWPGYEWLRNAAKK

SEQ ID NO: 22 - protein, 33 aa, Artificial
  Synthetic
SLEEEWAQIQCEVWGRGCPSESFYDWFEAQLHA

SEQ ID NO: 23 - protein, 33 aa, Artificial
  Synthetic
SLEEEWAQIQCEVWGRGCPSEDFYDWFEEQLHN

SEQ ID NO: 24 - protein, 31 aa, Artificial
  Synthetic
SLEEEWAQIQCEVWGRGCPSESFYDWFERQL

SEQ ID NO: 25 - protein, 31 aa, Artificial
  Synthetic
SLEEEWAQIECEVYGRGCPSEGFYNAIELLS

SEQ ID NO: 26 - protein, 34 aa, Artificial
  Synthetic
HGLEEEWAQIQCEVWGRGCPSESFYDWFEAQLHA

SEQ ID NO: 27 - protein, 32 aa, Artificial
  Synthetic
SLEEEWAQIQCEVWGRGCPSESFYDWFERQLY

SEQ ID NO: 28 - protein, 33 aa, Artificial
  Synthetic
SLEEEWAQIQAEVWGRGAPSESFYDWFEAQLHA

SEQ ID NO: 29 - protein, 32 aa, Artificial
  Synthetic
SLEEEWAQIQCEVWGRGCQRPEPFYDWFERQL

SEQ ID NO: 30 - protein, 32 aa, Artificial
  Synthetic
HGLEEEWAQHEEDVYHPPAESFYDWFEAQLHA

SEQ ID NO: 31 - protein, 32 aa, Artificial; identical to SEQ ID NO: 30
  Synthetic

SEQ ID NO: 32 - protein, 33 aa, Artificial
  Synthetic
SLEEEWAQIQCEVWGRGCPSEAFYDWFAEQLDD

SEQ ID NO: 33 - protein, 34 aa, Artificial
  Synthetic
SLEEEWAQIEAEVWGRGAPPSESFYDWFERQLGY

SEQ ID NO: 34 - protein, 18 aa, Artificial
  Synthetic
SLEEEWAQIECEVYGRGC

SEQ ID NO: 35 - protein, 9 aa, Artificial
  Synthetic
FYDWFERQL

SEQ ID NO: 36 - protein, 33 aa, Artificial
  Synthetic
SLEEEWAQIQCEVWGRGCPSEGFYDWFLQDDHV

SEQ ID NO: 37 - protein, 33 aa, Artificial
  Synthetic
SLEEEWAQIQCEVWGRGCPSEVFYDWFYFDDHD

SEQ ID NO: 38 - protein, 34 aa, Artificial
  Synthetic
SLEEEWAQIQCEVWGRGCQRPEPFYDWFAEQLDD

SEQ ID NO: 39 - protein, 31 aa, Artificial
  Synthetic
SLEEEWAQIQCEVWGRGCKSESFYDWFERQL

SEQ ID NO: 40 - protein, 39 aa, Artificial
  Synthetic
SLEEEWAQIQCEVWGRGCPSGGSTFEDYLHNVVFVPRPS

SEQ ID NO: 41 - protein, 31 aa, Artificial
  Synthetic
SLEEEWAQIECEVYGRGCPSESFYDWFERQL

SEQ ID NO: 42 - protein, 31 aa, Artificial; identical to SEQ ID NO: 41
  Synthetic

SEQ ID NO: 43 - protein, 33 aa, Artificial
  Synthetic
SLEEEWAQIQCEVWGRGCPSEAFYDWFHEQLDD

SEQ ID NO: 44 - protein, 24 aa, Artificial
  Synthetic
QIQCEVWGRGCPSESFYDWFERQL

SEQ ID NO: 45 - protein, 31 aa, Artificial
  Synthetic
SLEEEWAQIECEVYGRGCPSESFYHWFERQL

SEQ ID NO: 46 - protein, 31 aa, Artificial
  Synthetic
SLEEEWAQIQCEVWGRGCPSESFYGWFERQL

SEQ ID NO: 47 - protein, 31 aa, Artificial
  Synthetic
SLEEEWAQIQCEVWGRGCPSESFYGWFQAQL

SEQ ID NO: 48 - protein, 129 aa, Artificial
  Synthetic
GSSHHHHHHSSGLVPRGSHMQIFVKTLTGKTITLEVEPSDTIENVKAKIQDKEGIPPDQQRLIFAGKQLEDGRTLSDYNIQKESTLHLVLRLRGGIDKSLEEEWAQIECEVYGRGCPSESFYDWFERQL

SEQ ID NO: 49 - protein, 19 aa, Artificial
  Synthetic
GSLDESFYDWFERQLGKKY

SEQ ID NO: 50 - protein, 32 aa, Artificial
  Synthetic
SLEEEWAQIQCEVWGRGCPSESFYDWFERQLG

SEQ ID NO: 51 - protein, 31 aa, Artificial
  Synthetic
SLEEEWAQIQAEVWGRGAPSESFYDWFERQL

SEQ ID NO: 52 - protein, 31 aa, Artificial
  Synthetic
SLEEEWAQIQCEVWGRGCPSEGFYNAIELLS

SEQ ID NO: 53 - protein, 31 aa, Artificial
  Synthetic
SLEHEWAQIHCEVYGRGCPSESFYHWFERQL

SEQ ID NO: 54 - protein, 16 aa, Artificial
  Synthetic
GSLDESFYDWFERQLG

SEQ ID NO: 55 - protein, 21 aa, Artificial
  Synthetic
SLEEEWAQIQCEVWGRGCPSY

SEQ ID NO: 56 - protein, 25 aa, Artificial
  Synthetic
VQDDCRGRPCGDADSFYEWFDQQAM

SEQ ID NO: 57 - protein, 19 aa, Artificial
  Synthetic
SLEEEWAQIQCEVWGRGCP

SEQ ID NO: 58 - protein, 18 aa, Artificial
  Synthetic
SLEEEWAQIQCEVWGRGC

SEQ ID NO: 59 - protein, 11 aa, Artificial
  Synthetic
GFYGWFNAQLA

SEQ ID NO: 60 - protein, 31 aa, Artificial
  Synthetic
SLEEHWAQVECEVYGRGCPSESFYDWFERQL

SEQ ID NO: 61 - protein, 31 aa, Artificial
  Synthetic
SLEEEWGQVECEVYGRGCPSESFYDWFERQL

SEQ ID NO: 62 - protein, 30 aa, Artificial
  Synthetic
SLEEEWAQVECEVYGRCPSESFYDWFERQL

SEQ ID NO: 63 - protein, 31 aa, Artificial
  Synthetic
SLEEEWAQVECEVYGRNCPSESFYDWFERQL

SEQ ID NO: 64 - protein, 31 aa, Artificial
  Synthetic
SLEEEWAQVECEVYQRGCPSESFYDWFERQL

SEQ ID NO: 65 - protein, 31 aa, Artificial
  Synthetic
SLEEEWAQKECEVYGRGCPSESFYDWFERQL

SEQ ID NO: 66 - protein, 31 aa, Artificial
  Synthetic
SLEEEWAQRECEVYGRGCPSESFYDWFERQL

SEQ ID NO: 67 - protein, 31 aa, Artificial
  Synthetic
SLEEEWAQHECEVYGRGCPSESFYDWFERQL

SEQ ID NO: 68 - protein, 31 aa, Artificial
  Synthetic
SLEEYWAQVECEVYGRGCPSESFYDWFERQL

SEQ ID NO: 69 - protein, 31 aa, Artificial
  Synthetic
SLEEVWAQVECEVYGRGCPSESFYDWFERQL

SEQ ID NO: 70 - protein, 31 aa, Artificial
  Synthetic
SLEEEWYQVECEVYGRGCPSESFYDWFERQL

SEQ ID NO: 71 - protein, 31 aa, Artificial
  Synthetic
SLEEEWAQVECEVYGRHCPSESFYDWFERQL

SEQ ID NO: 72 - protein, 31 aa, Artificial
  Synthetic
SLEEEWAQVECEVYGRVCPSESFYDWFERQL

SEQ ID NO: 73 - protein, 31 aa, Artificial
  Synthetic
SLEEEWAQVECEVYGRFCPSESFYDWFERQL

SEQ ID NO: 74 - protein, 31 aa, Artificial
  Synthetic
SLEEEWAQVECEVYHRGCPSESFYDWFERQL

SEQ ID NO: 75 - protein, 31 aa, Artificial
  Synthetic
SLEEEWAQVECEVYERGCPSESFYDWFERQL

SEQ ID NO: 76 - protein, 31 aa, Artificial
  Synthetic
SLEEHWHQVECEVYGRGCPSESFYDWFERQL

SEQ ID NO: 77 - protein, 31 aa, Artificial
  Synthetic
SLEEHWHQVECEVYHRHCPSESFYDWFERQL

SEQ ID NO: 78 - protein, 31 aa, Artificial
  Synthetic
SLEEEWAQVECEVYGRGCPSESFYDWFERQL

SEQ ID NO: 79 - protein, 31 aa, Artificial
  Synthetic
SLEEEWAQIQEEVWGRGEPSESFYDWFERQL

SEQ ID NO: 80 - protein, 31 aa, Artificial; identical to SEQ ID NO: 41
  Synthetic

SEQ ID NO: 81 - protein, 31 aa, Artificial; identical to SEQ ID NO: 41
  Synthetic

SEQ ID NO: 82 - protein, 31 aa, Artificial; identical to SEQ ID NO: 24
  Synthetic

SEQ ID NO: 83 - protein, 31 aa, Artificial
  Synthetic
SLEEEWAQIECEVWGRGCPSEGFYNAIELLS

SEQ ID NO: 84 - protein, 32 aa, Artificial; identical to SEQ ID NO: 8
  Synthetic

SEQ ID NO: 85 - protein, 16 aa, Artificial
  Synthetic
GSLDESFYDWFERQLG

SEQ ID NO: 86 - protein, 31 aa, Artificial; identical to SEQ ID NO: 24
  Synthetic

SEQ ID NO: 87 - protein, 17 aa, Artificial
  Synthetic
YEEESFYDWFERQLGEE

SEQ ID NO: 88 - protein, 20 aa, Artificial
  Synthetic
SLEEEWAQIQCEVWGRGCPS

SEQ ID NO: 89 - protein, 17 aa, Artificial
  Synthetic
YEEESFYGWFERQLGEE

SEQ ID NO: 90 - protein, 32 aa, Artificial
  Synthetic
SLEEEWAQIQCEVWGRGCPSESFYGWFERQLG

SEQ ID NO: 91 - protein, 32 aa, Artificial
  Synthetic
SLEEEWAQVECEVWGRHCPSESFYGWFERQLG

SEQ ID NO: 92 - protein, 37 aa, Artificial
  Synthetic
SLEEEWAQIQCEVWGRGCPSESFYDWFERQLKKKKKK

SEQ ID NO: 93 - protein, 37 aa, Artificial
  Synthetic
KKKKKKSLEEEWAQIQCEVWGRGCPSESFYDWFERQL

SEQ ID NO: 94 - protein, 17 aa, Artificial
  Synthetic
EEESFYDWFERQLGEEY

SEQ ID NO: 95 - protein, 49 aa, Artificial
  Synthetic
SLEEEWAQIQCEVWGRGCPSQILKELEESSFRKTFEDYLHNVVFVPRPS

SEQ ID NO: 96 - protein, 32 aa, Artificial
  Synthetic
SLEEEWAQIQCEVWGRPCPSESFYGWFERQLG

SEQ ID NO: 97 - protein, 32 aa, Artificial
  Synthetic
SLEEEWAQIQCEVWGRGCPSESFYGWFEAQLG

SEQ ID NO: 98 - protein, 32 aa, Artificial
  Synthetic
SLEEEWAQIQCEVWGRGCPSESFYDWFERQLK

SEQ ID NO: 99 - protein, 31 aa, Artificial; identical to SEQ ID NO: 24
  Synthetic

SEQ ID NO: 100 - protein, 32 aa, Artificial; identical to SEQ ID NO: 90
  Synthetic

SEQ ID NO: 101 - protein, 32 aa, Artificial; identical to SEQ ID NO: 90
  Synthetic

SEQ ID NO: 102 - protein, 32 aa, Artificial; identical to SEQ ID NO: 90
  Synthetic

SEQ ID NO: 103 - protein, 32 aa, Artificial; identical to SEQ ID NO: 90
  Synthetic

SEQ ID NO: 104 - protein, 32 aa, Artificial; identical to SEQ ID NO: 90
  Synthetic

SEQ ID NO: 105 - protein, 32 aa, Artificial; identical to SEQ ID NO: 90
  Synthetic

SEQ ID NO: 106 - protein, 32 aa, Artificial
  Synthetic
SLEEEWAQIQCEVWGRGCPSESXYGWFERQLG

SEQ ID NO: 107 - protein, 32 aa, Artificial; identical to SEQ ID NO: 106
  Synthetic

SEQ ID NO: 108 - protein, 32 aa, Artificial; identical to SEQ ID NO: 106
  Synthetic

SEQ ID NO: 109 - protein, 32 aa, Artificial; identical to SEQ ID NO: 106
  Synthetic

SEQ ID NO: 110 - protein, 32 aa, Artificial; identical to SEQ ID NO: 106
  Synthetic

SEQ ID NO: 111 - protein, 32 aa, Artificial; identical to SEQ ID NO: 106
  Synthetic

SEQ ID NO: 112 - protein, 32 aa, Artificial; identical to SEQ ID NO: 106
  Synthetic

SEQ ID NO: 113 - protein, 32 aa, Artificial; identical to SEQ ID NO: 106
  Synthetic

SEQ ID NO: 114 - protein, 32 aa, Artificial
  Synthetic
SLEHEWAQIQCEVWGRGCPSEPFYGWFLAQLG

SEQ ID NO: 115 - protein, 32 aa, Artificial
  Synthetic
SLEHEWAQIQCEVWGRGCPSEPFYGWFERQLG

SEQ ID NO: 116 - protein, 32 aa, Artificial
  Synthetic
SLEHEWAQIHCEVWGRGCPSESFYHWFERQLG

SEQ ID NO: 117 - protein, 18 aa, Artificial
  Synthetic
SLEEEWAQIQCEVWGRGC

SEQ ID NO: 118 - protein, 10 aa, Artificial
  Synthetic
FYGWFERQLG

SEQ ID NO: 119 - protein, 32 aa, Artificial
  Synthetic
SLEEEWAQIQCEVWGXGCPSESFYGWFERQLG

SEQ ID NO: 120 - protein, 18 aa, Artificial
  Synthetic
SLEXEWXQIQCEVWGRGC

SEQ ID NO: 121 - protein, 10 aa, Artificial
  Synthetic
FYGWFEXQLG

SEQ ID NO: 122 - protein, 32 aa, Artificial
  Synthetic
SLEHEWAQIQCEVWGRGCPSESFYGWFERQLG

SEQ ID NO: 123 - protein, 32 aa, Artificial
  Synthetic
SLEHEWAQIQCEVWGRGCPSESFYGWFHEQLG

SEQ ID NO: 124 - protein, 32 aa, Artificial
  Synthetic
SLEHEWAQIQCEVWGRPCPSEPXYGWXHEQLG

SEQ ID NO: 125 - protein, 129 aa, Artificial
  Synthetic
GSSHHHHHHSSGLVPRGSHMQIFVKTLTGKTITLEVEPSDTIENVKAKIQDKEGIPPDQQRLIFAGKQLEDGRTLSDYNIQKESTLHLVLRLRGGIDKSLEEEWAQIQCEVWGRGCPSESFYDWFERQL

SEQ ID NO: 126 - protein, 32 aa, Artificial
  Synthetic
SLEHEWAQIQCEVWGRPCPSEPFYGWFHEQLG

SEQ ID NO: 127 - protein, 32 aa, Artificial
  Synthetic
SLEHEWAQIQCEVWGRPCPSEPXYGWFHEQLG

SEQ ID NO: 128 - protein, 32 aa, Artificial
  Synthetic
SLEHEWAQIQCEVWGRPCPSEPFYGWXHEQLG

SEQ ID NO: 129 - protein, 34 aa, Artificial
  Synthetic
SLEHEWXQIQCEVWGRPCPSEPXYGWFHEQLGPP

SEQ ID NO: 130 - protein, 32 aa, Artificial
  Synthetic
SLEEEWAQIQCEVWGRGCPXESFYGWFERQLG

SEQ ID NO: 131 - protein, 32 aa, Artificial; identical to SEQ ID NO: 130
  Synthetic

SEQ ID NO: 132 - protein, 34 aa, Artificial; identical to SEQ ID NO: 129
  Synthetic

SEQ ID NO: 133 - protein, 34 aa, Artificial
  Synthetic
SLEEEWXQIQCEVWGRPCPSEPXYGWFHEQLGPP

SEQ ID NO: 134 - protein, 34 aa, Artificial
  Synthetic
SLEXEWXQIQCEVWGRPCPSEPXYGWFHEQLGPP

SEQ ID NO: 135 - protein, 31 aa, Artificial
  Synthetic
SLEEEWXQIQCEVWGRGCPSESFYDWFERQL

SEQ ID NO: 136 - protein, 31 aa, Artificial
  Synthetic
SLEEEWAXIQCEVWGRGCPSESFYDWFERQL

SEQ ID NO: 137 - protein, 31 aa, Artificial
  Synthetic
SLEEEWAQIQCEVWGRGCPSESFYXWFERQL

SEQ ID NO: 138 - protein, 31 aa, Artificial
  Synthetic
SLEEEWAQIQCEVWGRGCPSESFYDWFXRQL

SEQ ID NO: 139 - protein, 31 aa, Artificial
  Synthetic
SLEEEWAPIQCEVWGRGCPSESFYDWFERQL

SEQ ID NO: 140 - protein, 31 aa, Artificial
  Synthetic
SLEEEWAQIQCEVWGRGCPSESFYDWFPRQL

SEQ ID NO: 141 - protein, 31 aa, Artificial
  Synthetic
SLEEEWAQIQCEVWGRGCPSESFYPWFERQL

SEQ ID NO: 142 - protein, 32 aa, Artificial
  Synthetic
SLEHEWXQIQCEVWGRPCPSEPXYGWFHEQLG

SEQ ID NO: 143 - protein, 32 aa, Artificial
  Synthetic
SLEHEWXQIQCEVWGRPCPSEPFYGWXHEQLG

SEQ ID NO: 144 - protein, 31 aa, Artificial
  Synthetic
SLEEEWAKIQCEVWGRGCPSESFYDWFERQL

SEQ ID NO: 145 - protein, 32 aa, Artificial; identical to SEQ ID NO: 130
  Synthetic

SEQ ID NO: 146 - protein, 32 aa, Artificial; identical to SEQ ID NO: 130
  Synthetic

SEQ ID NO: 147 - protein, 32 aa, Artificial
  Synthetic
SLEHEWXQIQCEVWGRPCPXEPXYGWFHEQLG

SEQ ID NO: 148 - protein, 32 aa, Artificial; identical to SEQ ID NO: 130
  Synthetic

SEQ ID NO: 149 - protein, 32 aa, Artificial; identical to SEQ ID NO: 147
  Synthetic

SEQ ID NO: 150 - protein, 32 aa, Artificial; identical to SEQ ID NO: 147
  Synthetic

SEQ ID NO: 151 - protein, 34 aa, Artificial
  Synthetic
SLEEEWXQIQCEVWGRPCPSEPXYGWXHEQLGPP

SEQ ID NO: 152 - protein, 34 aa, Artificial
  Synthetic
SLEEEWXQIQCEVWGRPCPSEPXYGWFXEQLGPP

(19) United States
(12) Patent Application Publication
(10) Pub. No.: US 2010/0298213 A1
(43) Pub. Date: Nov. 25, 2010

(54) Pharmaceutically Active Insulin Receptor-Modulating Molecules

(75) Inventor: Lauge Schaffer, Copenhagen Ø (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsværd (DK)

(21) Appl. No.: 11/660,846

(22) PCT Filed: Aug. 19, 2005
(86) PCT No.: PCT/EP2005/054095

§ 371 Date: Sep. 27, 2007

Related U.S. Application Data
(60) Provisional application No. 60/603,513, filed on Aug. 20, 2004.
(60) Provisional application No. 60/612,476, filed on Sep. 23, 2004.

(51) Int. Cl. A61K 38/17; C07K 14/705; C12P 21/06; C07H 21/04

(52) U.S. Cl. 514/6.7; 435/69.1; 435/320.1; 435/325; 530/324; 530/325; 536/23.5

(57) ABSTRACT

The invention described herein provides novel pharmaceutically active molecules (including novel peptide derivatives and peptides) that bind to an insulin receptor; compositions comprising such molecules; methods of modulating insulin receptor activity comprising the delivery of such molecules and related insulin-binding molecules (e.g., in the context of treating and/or preventing insulin receptor-related diseases such as diabetes); nucleic acids encoding such peptides; vectors and host cells comprising such nucleic acids; and methods of producing such molecules and compositions.

### FIELD OF THE INVENTION

The invention described here pertains to novel and useful pharmaceutical active molecules and related compositions and methods.

### BACKGROUND OF THE INVENTION

Insulin is a potent metabolic and growth promoting hormone that acts on cells to stimulate glucose, protein, and lipid metabolism, as well as RNA and DNA synthesis. A well-known effect of insulin is the regulation of glucose levels in the body. This effect occurs predominantly in liver, fat, and muscle tissue. In the liver, insulin stimulates glucose incorporation into glycogen and inhibits the production of glucose. In muscle and fat tissue, insulin stimulates glucose uptake, storage, and metabolism. Defects in glucose utilization are very common in the population, giving rise to diabetes.

Insulin initiates signal transduction in target cells by binding to a cell surface insulin receptor (IR). The human IR is a glycoprotein having molecular weight of 350-400 kDa (depending of the level of glycosylation). It is synthesized as a single polypeptide chain and proteolytically cleaved to yield a disulfide-linked α-β insulin monomer. Two α-β monomers are linked by disulfide bonds between the α-subunits to form a dimeric form of the receptor (β-α-α-β-type configuration). A human IR α-subunit typically is comprised of 723 amino acids, and it can be divided into two large homologous domains, L1 (amino acids 1-155) and L2 (amino acids 313-468), separated by a cysteine rich region (amino acids 156-312) (Ward et al., 1995, Prot. Struct. Funct. Genet. 22:141-153). Many determinants of insulin binding seem to reside in the α-subunit of the human IR. The human IR appears to be in dimeric form in the absence of ligand.

A binding model for IRs such as the human IR has been presented. This model proposes an IR comprising two insulin binding sites positioned on two different surfaces of the receptor molecule, such that each alpha-subunit is involved in insulin binding. In this way, activation of the insulin receptor is believed to involve cross-connection of the α-subunits by insulin.

### DETAILED DESCRIPTION OF THE INVENTION

The invention described here provides a number of novel and useful pharmaceutically active IR-binding molecules. These molecules include IR-binding peptides (IRBPs), including IRBP derivatives (or IRBPDs), and can be characterized on, among other things, the basis of comprising one or more IR-binding amino acid sequences (IRBAASs) explicitly provided herein or, in certain aspects, in one or more of the following patent documents: US Patent Application Publication Nos. 20030236190 and 20030195147; U.S. patent application Ser. No. 09/538,038; and International Patent Applications WO 01/72771, WO 03/027246, and WO 03/070747. These documents are collectively referred to herein as the “prior patent documents”. The invention also provides, for example, related compositions, such as nucleic acids encoding IRBPs; vectors and host cells comprising such nucleic acids; and compositions comprising such IR-binding molecules in combination with one or more pharmaceutically acceptable excipients. Methods of using such compositions and molecules to induce, promote, and/or enhance physiological responses, such as binding an IR in vivo, also are provided.

The phrase “pharmaceutically active” means that the IR-binding molecules provided by and/or used in the methods of this invention are able to exhibit biological activity (e.g., IR binding, IR activation, glucose reduction, etc.) in a mammalian host. Although pharmaceutically active IRBPs and IRBP compositions are an advantageous aspect of the invention, non-pharmaceutically active IRBPs and IRBP compositions also are provided by the invention, which can be useful in, e.g., diagnostic applications and/or the design of pharmaceutically useful molecules, such as by the methods described in the prior patent documents.

For sake of convenience the terms peptide, protein, and polypeptide are to be construed as providing support for one another herein(and thus, in a way, being interchangeable with one another herein), unless otherwise stated or clearly contradicted by context. For example, an individual reference to a “protein” herein should be construed as also providing equivalent literal support for an essentially identical aspect of the invention involving a “peptide” (a single chain protein of from 3 to about 50 amino acid residues) or “polypeptide” (a single chain protein of > about 50 amino acid residues in length), provided that such an understanding is reasonable and not contradicted. This is not to imply that these amino acid peptide bond polymeric molecules are not significantly different from one another in certain aspects (e.g., in terms of formulation for oral delivery). Thus, terms such as “protein” and “peptide” used individually herein should generally be understood as referring to any suitable amino acid-based oligomeric/polymeric molecule of any suitable size and composition (e.g., with respect to the number of associated chains comprised thereby and number of individual amino acid residues contained therein), as well as origin (e.g., obtained by recombinant expression, isolation from natural sources, production by solid phase synthesis, etc.). Typically, a peptide in this invention refers to a single primarily peptide bond-linked amino acid polymer containing molecule (e.g., a single amino acid chain or a derivative thereof).

It also should be understood that unless otherwise stated or clearly contradicted by context, a “protein” in the context of this invention can comprise non-essential, non-naturally occurring (or otherwise unusual), and/or non-L amino acid residues. Non-limiting examples of unusual amino acid residues that can be comprised in a derivative include, for example, 2-aminoadipic acid; 3-Aminoadipic acid; β-Alanine; β-aminopropionic acid; 2-Aminobutyric acid; 4-Aminobutyric acid; 6-Aminocaproic acid; 2-Aminoheptanoic acid; 2-Aminoisobutyric acid; 3-Aminoisobutyric acid, 2-Aminopimelic acid; 2,4-Diaminobutyric acid; Desmosine; 2,2′-Diaminopimelic acid; 2,3-Diaminopropionic acid; N-Ethylglycine; N-Ethylasparagine; Hydroxylysine; allo-Hydroxylysine; 3-Hydroxyproline; 4-Hydroxyproline; Isodesmosine; allo-Isoleucine; N-Methylglycine; N-Methylisoleucine; 6-N-Methyllysine; N-Methylvaline; Norvaline; Norleucine; and Ornithine. Additionally advantageous unusual amino acids relevant to particular aspects of the invention are described further elsewhere herein. It should be understood that the terms “peptide” and “protein” as used herein also encompass derivatized proteins, which are further described elsewhere herein, unless otherwise stated or clearly contradicted by context. Protein derivatives and proteins can be associated with significantly different features, however, and also can be considered unique aspects of the invention. In other words, the “inclusion” of derivatives in the broadest meaning of the term “protein” is done for purposes of convenience in describing the various features of this invention, rather than to imply any sort of equivalence between such molecules.

IRBPs and IRBAASs are characterized by binding an IR. Unless otherwise stated, aspects of this invention are described with reference to the human IR. However, it should be understood that IRBPs and IRBAASs provided by this invention also or alternatively may bind to other IRs, such as a mouse IR, rat IR, primate IR, pig IR, dog IR, etc.

A. IRBPS

As already mentioned, in one aspect, the invention provides novel and useful IRBPs. In another aspect, the invention provides new and useful methods of using IRBPs disclosed herein and/or in the prior patent documents. In some cases, the invention provides IRBPs derived from insulin receptor-binding amino acid sequences and/or peptides disclosed in the prior patent documents.

IRBPs can be prepared by any suitable method. For example, IRBPs, particularly non-derivative IRBPs, can be produced as fusion proteins in any suitable expression system. Methods and principles relevant to the production of recombinant fusion proteins are very well known in the art and need not be discussed in detail here. Standard peptide synthesis can be used to generate IRBPs as well. Such recombinantly produced or synthesized peptides can further be subjected to derivation, conjugation, multimerization, etc. to form more complicated molecules within the scope of this invention. Multivalent IRBPs and IRBP fusion proteins also can be generated by conventional chemical linkage of amino acid chains and/or other moieties/substituent molecules. Again, such methods and the principles related thereto are well characterized in the art. IRBPs likewise can be purified by any suitable technique. For example, IRBP fusion proteins comprising particular purification “tags” (purification facilitating sequences or moieties) can be generated by known methods and used to obtain such molecules. For direct purification, methods such as differential electrophoresis, chromatography, centrifugation also can be used as can affinity (e.g., antibody-based) methods directed to the characteristics of a non-fusion protein IRBP. A number of such techniques are specifically described with respect to the production and purification of IRBPs in the prior patent documents.

In the remainder of this Section A, general features of the IRBPs of the invention are first described followed by a description of exemplary classes of IRBPs provided by the invention and illustrative specific examples of such IRBPs.

1. General Features

IRBPs can be characterized on the basis of their ability to specifically bind to one or both sites of IR. In general, an IRBAAS binds to either Site 1 or Site 2 of an IR. However, multivalent IRBPs and, more particularly, multivalent multispecific IRBPs are also provided by the invention. Such IRBPs, which are further described elsewhere herein, generally comprise at least one Site 1-specific IRBAAS and at least one Site 2-specific IRBAAS.

IRBPs of the invention typically are capable of activating the insulin signaling pathway, as shown by, e.g., increased in vitro lipogenesis and by decreased glucose levels after intravenous (i.v. or IV) administration to pigs and anaesthetized rats. IRBPs can, for example, can increase in vitro lipogenesis in insulin receptor-bearing adipocytes about 10% as effective as human insulin (or more) (e.g., at least about 15% as effective as human insulin), about 25% as effective as human insulin (or more), about 33% as effective as human insulin (or more), about 50% as effective as human insulin (or more), about 60% as effective as human insulin (or more). IRBPs can dose-dependently increase whole-body glucose disposal, with potency in the same range as normal insulin.

Typically, the IRBPs of the invention are peptides of about 70 amino acids or less in length, such as less than about 60 amino acids in length, such as about 50 amino acids or less in length (e.g., about 30-50 amino acids in length).

Surprisingly, IRBAASs relevant to the IRBPs of this invention do not exhibit significant similarity with the amino acid sequence of insulin over more than a few amino acid residues in any particular region of the respective amino acid sequences thereof. The differences in composition of the IRBAAS comprised in the IRBPs of the invention with respect to insulin are associated with various biological characteristics that further serve to distinguish the IRBPs from insulins.

In one exemplary aspect, the invention provides IRBPs having improved stability towards digestive mammalian (e.g., human) enzymes, such as pepsin, trypsin, chymotrypsin, elastase, and/or carboxypeptidase A. In particular aspects, the invention provides IRBPs that have at least about 50-fold greater stability, at least about 100-fold greater stability, at least about 150-fold greater stability, or even at least about 200-fold greater stability to one or more of such proteolytic digestive enzymes relative to the stability exhibited to one or more of these enzymes by previously described IRBP S597 (see the prior patent documents for a description of S597). The phrase “50-gold greater stability” means that the relevant enzyme takes 50 times longer to degrade the relevant peptide at a target site as compared to the time it takes to degrade the control peptide (here, S597). In one aspect, the stability is attributed, at least in part, to the presence of one or more unusual amino acids or moieties that promote enzymatic degradation resistance. In this respect, the invention provides IRBP derivatives comprising one or more degradation resistance-promoting unusual amino acid residues and/or organic moiety/group, wherein the presence of the residue(s) and/or group(s) increases the stability with respect to a substantially identical IRBP lacking the residue(s) and/or group(s) with respect to degradation by one or more of such enzymes.

In another exemplary aspect, IRBPs of the invention can be characterized by exhibiting IR phosphorylation levels that are significantly lower than that observed with IR binding by insulin. IRBPs also may be associated with a different IR phosphorylation profile than insulin.

b. IR Affinity

IRBPs of the invention generally exhibit high affinity for IR (K_{d} in the pM range). More particularly, IRBPs typically have or are expected to have an affinity (K_{d}) for IR of between about 10⁻⁷ to about 10⁻¹⁵ M, such as 10⁻⁸ to about 10⁻¹² M, or more particularly typically about 10⁻¹⁰ to about 10⁻¹² M.

In particular aspects, the invention provides IRBPs that have an affinity for the human insulin receptor (HIR) that is at least about 10%, about 20%, about 30%, about 40%, about 50% or more, such as about 60% or more, about 70% or more, about 80% or more, about 90% or more, or about 95% or more of the affinity exhibited by human insulin. In another aspect, the invention provides IRBPs that have an affinity for HIR that is about equal to the affinity exhibited by insulin for the HIR. In still another aspect, the invention provides IRBPs that exhibit greater affinity for the HIR than human insulin. For example, the invention provides IRBPs that exhibit about 110% or more, about 150% or more, about 175% or more, or even about 200% or more affinity for HIR than human insulin.

c. IR Selectivity/Specificity

Insulin-like growth factor-1 (IGF-1) and insulin competitively cross-react with IGF-1R and IR (see, e.g., L. Schäffer, 1994, Eur. J. Biochem. 221:1127-1132). Yet, despite 45% overall amino acid identity, insulin and IGF-1 bind only weakly to each other's receptor. The affinity of each peptide for the non-cognate receptor is about 3 orders of magnitude lower than that for the cognate receptor (see, e.g., Mynarcik, et al., 1997, J. Biol. Chem. 272:18650-18655). The differences in binding affinities may be partly explained by the differences in amino acids and unique domains which contribute to unique tertiary structures of ligands (Blakesley et al., 1996, Cytokine Growth Factor Rev. 7(2):153-9).

IRBPs typically are significantly more specific for IR than IGF-1R. Typically, the IR/IGF-1R binding affinity ratio exhibited by IRBPs is about 100 or more. In particular aspects, the invention provides IRBPs that exhibit a preference for IR over IGF-1R marked by an affinity ratio of at least about 1,000; at least about 5,000; at least about 10,000, or greater. In an even more particular aspect, the invention provides IRBPs that exhibit a preference for IR over IGF-1R marked by an affinity ratio of about 10,000 to about 100,000.

IRBPs also or alternatively can be characterized on the basis of their inability to activate IGF-1R. Thus, in one aspect, the invention provides IRBPs that are efficacious at IR activation but have little or no significant activity with respect to IGF-1R.

In yet a further aspect, the invention provides IRBS that also or alternatively are selective for the IR of a particular species as compared to other species. Thus, for example, in one aspect the invention provides IRBPs that exhibit a significant preference for human IR as compared to other mammalian IRs, such as rat IR and pig IR (e.g., a preference marked by an affinity ratio of at least about 1.1, 1.3, 1.4, 1.5, 1.6, 1.7, 1.8, 1.9, 2.0, 2.1, 2.2, 2.3, 2.4, 2.5, 2.6, 2.7, or higher).

In a further aspect still, the invention provides IRBPs that are selective for an isoform of a particular mammalian IR over another isoform. Isoforms of IRs are known to exist in several mammalian species. For example, HIR−11 and HIR+11 refer to the two isoforms of the human insulin receptor, without and with exon 11 respectively (such isoforms are apparently generated by an alternative splicing mechanism). These isoforms are also known as HIR A and HIR B. In an exemplary aspect of the invention, IRBPs that exhibit a preference for HIR−11 over HIR+11 are provided. In a different aspect, the invention provides IRBPs that exhibit a preference for HIR+11 over HIR−11. The invention similarly provides IRBPs that exhibit such selectivity for different isoforms in non-human mammalian species. HIR+11 and HIR−11, as well as IR isoforms of other species, are expressed at different levels in different tissues. Accordingly, the invention provides IRBPs that preferential associate with different tissue profiles when administered or otherwise delivered to a particular host, such as a human patient.

Selectivity, specificity, affinity, and avidity are concepts well understood in the art (the use of affinity herein may be considered to encompass avidity with respect to multivalent IRBPs), and several techniques are well known and readily available for assessing these measurements with respect to particular IRBPs (as compared to each other and/or different potential binding partners such as IRs of different species and/or an IR of a species as compared to an IGF-1R of the same or different species). Examples of such methods are described, e.g., in the prior patent documents.

d. Modulation of IR Activity

As already suggested, IRBPs typically have IR-modulating activity. Typically, IRBPs exhibit IR partial agonist or agonistic activity.

Also as mentioned above, IRBPs may in addition or alternatively to other characteristics, be characterized on the basis of their ability to lower blood glucose levels, which may be, for example, reflected by the results of a fat cell lipogenesis assay. IRBPs can in this context and other contexts exhibit at least about 10%, at least about 20%, at least about 30%, at least about 40%, at least about 50%, at least about 60%, or more (e.g., about 70-100%) of the blood glucose lowering abilities of human insulin in human insulin receptor-bearing cells.

e. Stability of IRBPs

In a particularly advantageous aspect, the invention provides IRBPs that exhibit greater stability against digestive enzymatic degradation than insulin or IRBPs described in the prior patent documents.

Thus, for example, in one aspect the invention provides an IRBP that is more resistant to degradation by at least one digestive enzyme (e.g., pepsin, chymotrypsin, both, or other similar enzyme) than insulin and that comprises at least one IRBAAS, which IRBAAS comprises at least one unusual and digestive enzyme degradation-resistant amino acid residue or other suitable and enzyme degradation-resistant chemical moiety. In one aspect, the unusual amino acid residue/moiety is selected from sarcosine (N-methylglycine); aminoisobutyric acid; diphenylalanine; N-methyl-phenylalanine; D-arginine; ornithine; 4-tertbutyl-phenylalanine; pyridylalanine; phenylglycine; homophenylalanine; cyclohexylalanine; 4-biphenylalanine; 2-aminoindane-2-carboxylic acid; N-Fmoc-8-amino-3,6-dioxaoctanoic acid; N-Fmoc-19-amino-5-oxo-3,10,13,16-tetraoxa-6-aza-nonadecanoic acid; C14-monocarboxylic acid; C20-dicarboxylic acid; polyethylene glycol (PEG) (e.g., a PEG with a molecular weight (MW) of about 5000); and 1-(4,4-dimethyl-2,6-dioxocyclohexylidene)ethyl. In another aspect, the invention provides a multivalent IRBP comprising at least two IRBAASs, wherein the IRBP comprises at least one unusual enzymatic degradation resistant amino acid residue or chemical moiety located between the IRBAASs. In another aspect, the invention provides IRBPs comprising such a degradation-resistant unusual residue or moiety located at a terminus of the IRBP. In a further facet, the invention provides a multivalent IRBP comprising at least two IRBAASs, wherein the IRBP comprises at least two of such degradation-resistant residues or moieties. The two or more residues/moieties can be located in a single IRBAAS or in the two or more IRBAAS. IRBS comprising any combination of degradation-resistant moieties and/or residues at (a) the termini of the IRBS, (b) between IRBAASs, and/or (c) in one or more IRBAASs, are provided by the invention. More specific examples of multivalent IRBPs comprising such degradation-resistant residues and moieties are described elsewhere herein.

2. IRBAAS Formulas

In order to better illustrate the invention, a description of particular types of IRBAASs and specific examples thereof is provided here. IRBPs can include any one or combination of such formulas (as further described below) or one or more of such formulas in combination with the various sequences and formulas provided in the prior patent documents.

a. Formulas 1a-1g

In one aspect, the invention provides an IRBAAS that consists or consists essentially of a sequence according to the formula Xaa₁ Tyr Xaa₃ Trp Xaa₅, wherein (a) Xaa₁, Xaa₅, or both represent either (i) degradation-resistant unusual amino acid residues or degradation-resistant chemical moieties or (ii) Phe residues, and (b) Xaa₃ is a degradation-resistant unusual amino acid residue, a non-amino acid residue degradation resistant chemical moiety, or any suitable other amino acid residue (Formula 1a). In a more particular aspect, the invention provides an IRBAAS that consists or consists essentially of a sequence according to the formula Xaa₁ Tyr Xaa₃ Trp Xaa₅ Xaa₆ Xaa₇ Xaa₈ Xaa₉, wherein Xaa₆ is any suitable amino acid residue (typically a residue other than Asp or Asn); Xaa₇ is any suitable residue; Xaa₈ is selected from Gln, Glu, Ala, and Lys; and Xaa₉ represents a hydrophobic amino acid (Formula 1b). In still a more particular aspect, the invention provides an IRBAAS that consists or consists essentially of a sequence according to the formula Xaa₁ Tyr Xaa₃ Trp Xaa₅ Glu Arg Gln Leu (SEQ ID NO: 1), wherein Xaa₁, Xaa₃, and Xaa₅ are defined as in Formula 1a (Formula 1c). In yet an even more specific aspect, the invention provides an IRBAAS that consists or consists essentially of a sequence according to the formula Xaa₁ Tyr Xaa₃ Trp Xaa₅ Glu Arg Gln Leu Gly (SEQ ID NO:2), wherein Xaa₁, Xaa₃, and Xaa₅ are defined as in Formula 1a (Formula 1d).

In another particular new aspect, the invention provides an IRBAAS that consists or consists essentially of a sequence according to the formula Xaa₁ Tyr Gly Trp Xaa₅ Glu Arg Gln Xaa₉ Gly (SEQ ID NO:3), wherein Xaa₁ is a Phe or degradation-resistant residue/moiety; Xaa₅ is a Phe or degradation-resistant moiety/residue; and Xaa₉ is any suitable residue (and typically a Leu) (Formula 1e).

In a further aspect, the invention provides an IRBAAS that consists or consists essentially of a sequence according to the formula Xaa₁ Tyr Xaa₃ Trp Xaa₅ Glu Arg Gln Leu Gly (SEQ ID NO:4), wherein Xaa₁ and Xaa₅ are defined as in Formula 1e, and Xaa₃ is a Gly or His residue (Formula 1f).

In still another exemplary aspect, the invention provides an IRBAAS that consists or consists essentially of a sequence according to the formula Xaa₁ Tyr Xaa₃ Trp Xaa₅ Xaa₆ Xaa₇ Xaa₈ Xaa₉ Xaa₁₀, wherein Xaa₁ is a Phe or degradation-resistant residue/moiety; Xaa₅ is a Phe or degradation-resistant moiety/residue; Xaa₃ is any suitable residue; Xaa₆-Xaa₈ are any suitable residues; Xaa₉ is any suitable residue or is missing; and Xaa₁₀ is a hydrophobic residue (Formula 1g). In a particular aspect, the invention provides IRBAASs that consist or consist essentially of a sequence according to Formula 1g wherein one or more (or all) of Xaa₆₋₈ and also or alternatively Xaa₉ (is present) are hydrophilic residues (e.g., Glu, Gln, Asp, Lys, or Arg residues). In one such aspect, most, or all, of such residues are hydrophilic. In another particular variant, the invention provides IRBAASs consisting or consisting essentially of a Formula 1g sequence wherein the sequence also or alternatively is characterized by Xaa₃ representing a degradation-resistant residue or moiety. An example of such an IRBAAS is an IRBAAS according to the more particular formula Phe Tyr Xaa₃ Trp Phe Glu Arg Gln Leu, wherein Xaa₃ represents an enzyme degradation-resistant amino acid residue or moiety. In still another variant of any of the foregoing IRBAAS aspects, Xaa₃ represents a residue selected from Glu, Gly, or His. In particular aspects, Xaa₁₀ is a Leu, Val, Met, Ile, or Gly residue. In an even more particular aspect, Xaa₁₀ represents either a Leu or Gly residue. In one aspect, a sequence according to any of the foregoing is provided wherein Xaa₉ and Xaa₁₀ both represent hydrophilic residues; such as, e.g., Leu and Gly, respectively.

b. Formula 2a

In another aspect, the invention provides an IRBAAS that consists or consists essentially of the sequence Ser Glu Gly Phe Tyr Asn Ala Ile Glu Leu Leu Ser (SEQ ID NO:5) (Formula 2a).

c. Formulas 6a-6g

In another aspect, the invention provides an IRBAAS that consists or consists essentially of a sequence according to the formula Xaa₁ Leu Glu Xaa₄ Glu Trp Xaa₇ Xaa₈ Xaa₉ Xaa₁₀ Xaa₁₁ Xaa₁₂ Val Tyr Xaa₁₅ Xaa₁₆ Xaa₁₇ Xaa₁₈ (SEQ ID NO:6), wherein Xaa₁, Xaa₄, Xaa₇, Xaa₈, Xaa₉, Xaa₁₀, Xaa₁₂, Xaa₁₅, Xaa₁₆, and Xaa₁₇ are any suitable amino acid residues and Xaa₁₁, Xaa₁₈, or both are any suitable residue other than Cys (Formula 6a). In a more particular aspect, the invention provides an IRBAAS that consists or consists essentially of a sequence according to formula 6a, wherein Xaa₁₁ is an Ala or Glu, Xaa₁₈ is an Ala or Glu, or both Xaa₁₁ and Xaa₁₈ are, independently, Ala or Glu residues (Formula 6b). In a particular aspect, Xaa₁₁ and/or Xaa₁₈ are Ala residues. In a further particular aspect, the invention provides an IRBAAS that consists essentially or consists of a sequence according to the formula Ser Leu Glu Glu Glu Trp Ala Gln Ile Glu Xaa₁₁ Glu Val Trp Gly Arg Gly Xaa₁₈ (SEQ ID NO:7), wherein Xaa11 and/or Xaa18 represent any suitable residue other than Cys (Formula 6c). In a more particular aspect, the invention provides an IRBAAS that consists or consists essentially of a sequence according to Formula 6c, wherein Xaa₁₁ and/or Xaa₁₈ represent Ala residues (Formula 6d).

In a further aspect, the invention provides an IRBAAS that consists or consists essentially of a sequence according to one or more of Formulas 6a-6d wherein the C-terminus of the sequence is joined to a C-terminal sequence according to the formula Xaa₁₉ Xaa₂₀ Xaa₂₁, wherein Xaa₂₁ is not a hydrophobic or aliphatic residue and Xaa₁₉ and Xaa₂₀ are any suitable residues. In a more particular aspect, Xaa₂₁ is a Glu residue. In yet another particular aspect, the C-terminal sequence is also or alternatively characterized by Xaa₁₉ representing a Pro residue, Xaa₂₀ representing a Ser residue, or both.

Examples of Formula 6d IRBAAS-containing IRBPs include peptides S574 (SLEEEWAQIEAEVWGRGAPSESFYDWFERQLG—SEQ ID NO:8) and S727 (Ac-SLEEEWAQIEAEVWGRGAPSESFYDWFERQLG-NH2—SEQ ID NO:9).

In a further aspect, the invention provides IRBAAS that consists or consists essentially of a sequence according to one or more of Formulas 6a-6d, typically with the inclusion of a C-terminal sequence as described in the preceding paragraph, wherein the N-terminal residue of the sequence (Xaa₁) is acylated and, more typically, acetylated. Typically, Xaa₁ represents an acetylated Ser residue. Peptide S727 is an example of an IRBP comprising such an IRBAAS.

Formula 1a-1g and similar IRBAASs are specific for IR Site 1, whereas Formulas 6a-6d, similar IRBAASs, and Formula 2a (and similar IRBAASs) bind to IR Site 2.

In yet another aspect, the invention provides an IRBAAS that consists or consists essentially of a sequence according to the formula Xaa₁ Leu Glu Xaa₄ Glu Trp Xaa₇ Xaa₈ Xaa₉ Xaa₁₀ Xaa₁₁ Xaa₁₂ Val Tyr Xaa₁₅ Xaa₁₆ Xaa₁₇ Xaa₁₈ (SEQ ID NO:10), wherein (a) Xaa₁₁ and/or Xaa₁₈ are Cys residues or other suitable amino acid residues and (b) one or more of Xaa₄, Xaa₇, Xaa₈, Xaa₁₅, and Xaa₁₇ represent degradation-resistant unusual amino acid residues and/or moieties (Formula 6e). In one aspect, such an IRBAAS comprises at least two degradation-resistant unusual residues or moieties.

In an additional exemplary aspect, the invention provides an IRBAAS that consists or consists essentially of a sequence according to the formula Ser Leu Glu Glu Glu Trp Ala Gln Ile Xaa₁₀ Xaa₁₁ Glu Val Trp Gly Arg Gly Xaa₁₈ (SEQ ID NO:11), wherein Xaa₁₀ is Glu or Gln and Xaa₁₁ and Xaa₁₈ are any suitable residues (Formula 6f). In one aspect, the invention provides IRBPS that comprise an IRBAAS wherein Xaa₁₁ and/or Xaa₁₈ are Cys residues. In a more particular aspect, both Xaa₁₁ and/or Xaa₁₈ are Cys residues. In an alternative aspect, both Xaa₁₁ and Xaa₁₈ are characterized as any suitable residue other than Cys residues. In a particular facet of such an aspect, Xaa₁₁ and/or Xaa₁₈ can be, for example, independently Ala or Glu residues.

In still another exemplary aspect, the invention provides an IRBAAS that consists or consists essentially of a sequence according to the formula Xaa₁ Leu Glu Xaa₄ Glu Trp Xaa₇ Xaa₈ Xaa₉ Xaa₁₀ Xaa₁₁ Xaa₁₂ Val Tyr Xaa₁₅ Xaa₁₆ Xaa₁₇ Xaa₁₈ (SEQ ID NO:12), wherein Xaa₁, Xaa₄, Xaa₇, Xaa₈, Xaa₉, Xaa₁₀, Xaa₁₂, Xaa₁₅, Xaa₁₆, and Xaa₁₇ are any suitable amino acid residues; Xaa₁₈ is Cys or a suitable residue other than Cys (e.g., Ala or Glu); and Xaa₁₁ is Cys or a suitable residue other than Cys (e.g., Ala or Glu) (Formula 6g). In one version, Xaa₁₈ is Cys. In one version, Xaa₁₀ also or alternatively is Glu or Gln.

In one exemplary aspect, the invention provides an IRBAAS that consists or consists essentially of a sequence according to one or more of Formulas 6a-6g, wherein the sequence is characterized as not forming internal Cys-Cys bonds, not comprising a Cys residue, and/or not forming a cyclic peptide conformation under typical physiological conditions.

4. Variants of IRBAASs

Also included within the scope of this invention are amino acid sequences containing acceptable amino acid residue substitutions, additions, or deletions and which bind to IR with the same or altered affinity.

In one such respect, the invention provides IRBP “fusion proteins” and variants related thereto. For example, sequence tags (e.g., FLAG® tags) or amino acids, such as one or more lysines, can be added to the peptide sequences of the invention (e.g., at the N-terminal or C-terminal ends). Sequence tags can be used for peptide purification or localization. Lysines can be added to a sequence to increase peptide solubility or to allow for biotinylation. Alternatively, amino acid residues located at the carboxy and amino terminal regions of IRBAASs that comprise sequence tags (e.g., FLAG® tags), or which contain amino acid residues that are not associated with a strong preference for a particular amino acid, may optionally be deleted providing for truncated sequences. Additional features of IRBP fusion proteins and related principles are described elsewhere herein and in the prior patent documents.

Variants also can include amino acid sequences in which one or more residues are modified (i.e., by phosphorylation, sulfation, acylation, PEGylation, etc.), and mutants comprising one or more modified residues with respect to a parent sequence. Amino acid sequences may also be modified with a label capable of providing a detectable signal, either directly or indirectly, including, but not limited to, radioisotope, fluorescent, and enzyme labels. Fluorescent labels include, for example, Cy3, Cy5, Alexa, BODIPY, fluorescein (e.g., FluorX, DTAF, and FITC), rhodamine (e.g., TRITC), auramine, Texas Red, AMCA blue, and Lucifer Yellow. Preferred isotope labels include ³H, ¹⁴C, 32 P, ³⁵S, ³⁶Cl, ⁵¹Cr, ⁵⁷Co, ⁵⁸Co, ⁵⁹Fe, ⁹⁰Y, ¹²⁵I, ¹³¹I, and ²⁸⁶Re. Preferred enzyme labels include peroxidase, β-glucuronidase, β-D-glucosidase, β-D-galactosidase, urease, glucose oxidase plus peroxidase, and alkaline phosphatase (see, e.g., U.S. Pat. Nos. 3,654,090; 3,850,752 and 4,016,043). Enzymes can be conjugated by reaction with bridging molecules such as carbodiimides, diisocyanates, glutaraldehyde, and the like. Enzyme labels can be detected visually, or measured by calorimetric, spectrophotometric, fluorospectrophotometric, amperometric, or gasometric techniques. Other labeling systems, such as avidin/biotin, Tyramide Signal Amplification (TSA™), are known in the art, and are commercially available (see, e.g., ABC kit, Vector Laboratories, Inc., Burlingame, Calif.; NEN® Life Science Products, Inc., Boston, Mass.).

Thus, in one aspect, the invention provides IRBPs that comprise one or more variant IRBAASs that differ from one or more parent IRBAASs specifically disclosed herein or in the prior patent documents (e.g., in the context of a sequence disclosed in the prior patent documents but modified by another principle described herein such as by N-terminal acetylation (or other acylation) and/or C-terminal amidation and/or by inclusion of degradation-resistant unusual amino acid residues and/or non-AA moieties) by the relative insertion, deletion, addition, or substitution of one or more amino acid residues. In another respect, the invention provides biologically active IRBP variants that comprise one or more IRBAASs that differ from the IRBAASs disclosed herein or the prior patent documents but exhibit at least about 60%, at least about 70%, at least about 80%, at least about 85%, at least about 90%, about 95%, or more identity to such parent IRBAASs. The invention encompasses such variants for all such sequences disclosed herein and in the prior patent documents (as modified by one or more of the various novel aspects described herein).

Typically, variants differ from “parent” sequences mostly through conservative substitutions; e.g., at least about 35%, about 50% or more, about 60% or more, about 70% or more, about 75% or more, about 80% or more, about 85% or more, about 90% or more, about 95% or more (e.g., about 65-99%) of the substitutions in the variant sequence are conservative amino acid residue replacements. In the context of this invention, conservative substitutions can be defined by substitutions within the classes of amino acids reflected in the following table:

TABLE 1

| | |
|---|---|
| Alcohol group-containing residues | S and T |
| Aliphatic residues | I, L, V, and M |
| “Aromatic” residues | F, H, W, and Y |
| Hydrophobic residues | A, C, F, G, H, I, L, M, R, T, V, |
| | W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, N, Q, R, S, |
| | P, and T |
| Flexible residues | E, Q, T, K, S, G, P, D, E, and R |

Substantial changes in function can be made by selecting substitutions that are less conservative than those shown in the defined groups, above. For example, non-conservative substitutions can be made which more significantly affect the structure of the peptide in the area of the alteration, for example, the alpha-helical, or beta-sheet structure; the charge or hydrophobicity of the molecule at the target site; or the bulk of the side chain. The substitutions which generally are expected to produce the greatest changes in the peptide's properties are those where 1) a hydrophilic residue, e.g., seryl or threonyl, is substituted for (or by) a hydrophobic residue, e.g., leucyl, isoleucyl, phenylalanyl, valyl, or alanyl; 2) a cysteine or proline is substituted for (or by) any other residue; 3) a residue having an electropositive side chain, e.g., lysyl, arginyl, or histidyl, is substituted for (or by) an electronegative residue, e.g., glutamyl or aspartyl; or 4) a residue having a bulky side chain, e.g., phenylalanine, is substituted for (or by) a residue that does not have a side chain, e.g., glycine. Accordingly, these and other nonconservative substitutions can be introduced into peptide variants where significant changes in function/structure is desired and such changes avoided where conservation of structure/function is desired.

Those skilled in the art will be aware of additional principles useful in the design and selection of peptide variants. For example, residues in surface positions of a peptide typically a strong preference for hydrophilic amino acids. Steric properties of amino acids can greatly affect the local structures that a protein adopts or favors. Proline, for example, exhibits reduced torsional freedom that can lead to the conformation of the peptide backbone being locked in a turn and with the loss of hydrogen bonding, often further resulting in the residue appearing on a surface loop of a protein. In contrast to Pro, Gly has complete torsional freedom about a main peptide chain, such that it is often associated with tight turns and regions buried in the interior of the protein (e.g., hydrophobic pockets). The features of such residues often limit their involvement in secondary structures. However, residues typically involved in the formation of secondary structures are known. For example, residues such as Ala, Leu, and Glu (amino acids without much bulk and/or polar residues) typically are associated with alpha-helix formation, whereas residues such as Val, Ile, Ser, Asp, and Asn can disrupt alpha helix formation. Residues with propensity for beta-sheet structure formation/inclusion include Val and Ile and residues associated with turn structures include Pro, Asp, and Gly. The skilled artisan can consider these and similar known amino acid properties in the design and selection of suitable peptide variants, such that suitable variants can be prepared with only routine experimentation.

Desirably, conservation in terms of hydropathic/hydrophilic properties also is substantially retained in a variant peptide as compared to a parent peptide (e.g., the weight class, hydropathic score, or both of the sequences are at least about 50%, at least about 60%, at least about 70%, at least about 75%, at least about 80%, at least about 85%, at least about 90%, at least about 95%, or more (e.g., about 65-99%) retained). Methods for assessing the conservation of the hydropathic character of residues/sequences are known in the art and incorporated in available software packages, such as the GREASE program available through the SDSC Biology Workbench (see also, e.g., Kyte and Doolittle et al., J. Mol. Biol. 157:105-132(1982); Pearson and Lipman, PNAS (1988) 85:2444-2448, and Pearson (1990) Methods in Enzymology 183:63-98 for a discussion of the principles incorporated in GREASE and similar programs).

It also is advantageous that structure of the variant peptide is substantially similar to the structure of the parent peptide. Methods for assessing similarity of peptides in terms of conservative substitutions, hydropathic properties, weight conservation, and similar considerations are described in e.g., International Patent Applications WO 03/048185, WO 03/070747, and WO 03/027246. Secondary structure comparisons can be made using the EBI SSM program (currently available at http://www.ebi.ac.uk/msd-srv/ssm/). Where coordinates of the variant are known they can be compared by way of alignment/comparison programs such as DALI pair alignment (currently available at http://www.ebi.ac.uk/dali/Interactive.html), TOPSCAN (currently available at http://www.bioinf.org.uk/topscan), COMPARER (currently available at http://www-cryst.bioc.cam.ac.uk/COMPARER/) PRIDE pair (currently available at http://hydra.icgeb.trieste.it/pride/pride.php?method=pair), PINTS (currently available at http://www.russell.embl.de/pints/), SARF2 (currently available at http://123d.ncifcrf.gov/run2.html), the Structural Alignment Server (currently available at http://www.molmovdb.org/align/), and the CE Calculate Two Chains Server (currently available at http://cl.sdsc.edu/ce/ce_align.html). Ab initio protein structure prediction methods can be applied, if needed, to the variant sequence, such as through the HMM-ROSETTA or MODELLER programs, to predict the structure for comparison with the parent sequence(s) molecule. Where appropriate other structure prediction methods, such as threading methods, also or alternatively can be used, to predict the structure of the variant and/or parent sequence proteins.

The retention of similar residues also or alternatively can be measured by a similarity score, as determined by use of a BLAST program (e.g., BLAST 2.2.8 available through the NCBI). Suitable variants typically exhibit at least about 45%, such as at least about 55%, at least about 65%, at least about 75%, at least about 85%, at least about 90%, at least about 95%, or more (e.g., about 70-99%) similarity to the parent peptide.

As discussed elsewhere herein, other points of variation/divergence between a variant and a parent can be acceptable (e.g., inclusion of non-naturally-occurring amino acids, derivatized amino acids, insertions, deletions, and extensions to the sequence, etc.) provided that such changes do not substantially impair the ability of the variant to bind IR as compared to the parent IRBAAS(s).

Identity in the context of amino acid sequences of the invention can be determined by any suitable technique, typically by a Needleman-Wunsch alignment analysis (see Needleman and Wunsch, J. Mol. Biol. (1970) 48:443-453), such as is provided via analysis with ALIGN 2.0 using the BLOSUM50 scoring matrix with an initial gap penalty of −12 and an extension penalty of −2 (see Myers and Miller, CABIOS (1989) 4:11-17 for discussion of the global alignment techniques incorporated in the ALIGN program). A copy of the ALIGN 2.0 program is available, e.g., through the San Diego Supercomputer (SDSC) Biology Workbench. Because Needleman-Wunsch alignment provides an overall or global identity measurement between two sequences, it should be recognized that target sequences which may be portions or subsequences of larger peptide sequences may be used in a manner analogous to complete sequences or, alternatively, local alignment values can be used to assess relationships between subsequences, as determined by, e.g., a Smith-Waterman alignment (J. Mol. Biol. (1981) 147:195-197), which can be obtained through available programs (other local alignment methods that may be suitable for analyzing identity include programs that apply heuristic local alignment algorithms such as FastA and BLAST programs). Further related methods for assessing identity are described in, e.g., International Patent Application WO 03/048185. The Gotoh algorithm, which seeks to improve upon the Needleman-Wunsch algorithm, alternatively can be used for global sequence alignments. See, e.g., Gotoh, J. Mol. Biol. 162:705-708 (1982).

Typically, advantageous sequence changes are those that (1) reduce susceptibility to proteolysis, (2) reduce susceptibility to oxidation, (3) alter binding affinity of the variant sequence (typically desirably increasing affinity), and/or (4) confer or modify other physicochemical or functional properties on the associated variant/analog peptide.

Amino acid sequence variations can result in an altered glycosylation pattern in the variant IRBAAS with respect to a parent IRBAAS. “Altering” in this context means removal of one or more glycosylation sites found in the parent IRBAAS and/or adding one or more glycosylation sites that are not present in the parent IRBAAS. Glycosylation is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are common recognition sequences for enzymatic attachment of the carbohydrate moiety to the asparagine side chain. Thus, the presence of either of these tripeptide sequences in a polypeptide can create a potential glycosylation site. O-linked glycosylation refers to the attachment of sugars such as N-aceylgalactosamine, galactose, or xylose to a hydroxyamino acid, most commonly serine or threonine, although 5-hydroxyproline or 5-hydroxylysine may also be used. Addition of glycosylation sites to a IRBAAS can be conveniently accomplished by altering the amino acid sequence of a variant IRBAAS with respect to the parent sequence such that it is caused to contain one or more of the above-described tripeptide sequences (for N-linked glycosylation sites) or other suitable glycosylation site. The alteration may also be made by, for example, the addition of, or substitution by, one or more serine or threonine residues to the sequence of the original IRBAAS (for O-linked glycosylation sites).

Amino acid sequence variants generally can be obtained by, for example, introducing appropriate nucleotide changes into an IRBAAS-encoding nucleic acid sequence (e.g., by site directed mutagenesis), by chemical peptide synthesis, or any other suitable technique. Such variants include, for example, variants differing by deletions from, insertions into, additions to (at either end of the parent sequence), and/or substitutions of, residues within the parent amino acid sequences. Any combination of deletions, insertions, additions, and substitutions can be made to arrive at a desired variant, provided that the variant possesses suitable characteristics for practice in the methods of the invention (e.g., retention of at least a substantial proportion of the parent sequences affinity for IR). There are a number of more sophisticated techniques available for obtaining variants including directed evolution, mutagenesis techniques, and the like.

Suitable variants can be assessed by screening assays described in the prior patent documents including, e.g., surface plasmon resonance affinity analysis (e.g., BIAcore™ analysis); IR autophosphorylation assays (e.g., holoenzyme phosphorylation assays); competition assays (e.g., Time-resolved fluorescence resonance energy transfer (TR-FRET) assays); and substrate phosphorylation assays (e.g., a HIR kinase assay); and intravenous blood glucose testing.

5. Multivalent and Multispecific IRBPs

As described above, in one aspect, the invention provides IRBPs that comprise two or more amino acid sequences which bind to one or more sites of IR (e.g., Site 1 or Site 2). Such multivalent IRBPs can be produced by standard fusion protein expression technology, chemical conjugation, or any other suitable technique for producing a multivalent IRBP. In one aspect, the invention provides a multivalent IRBP that comprises at least one Site 1-binding amino acid sequence and at least one site-2 binding amino acid sequence. Such IRBPs can be described as multispecific as well as multivalent. In another aspect, the invention provides a multivalent IRBP comprising two or more sequences that specifically bind to the same site on IR.

Multispecific IRBPs can be characterized on the basis of little or no competition between the Site 1 and Site 2 binding IRBAASs comprised therein.

Multivalent ligands may be prepared by either expressing amino acid sequences which bind to the individual sites separately and then covalently linking them together, or by expressing the multivalent ligand as a single amino acid sequence which comprises within it the combination of specific amino acid sequences for binding.

Various combinations of amino acid sequences may be combined to produce multivalent ligands having specific desirable properties. Thus, agonists may be combined with agonists, antagonists combined with antagonists, and agonists combined with antagonists. Combining amino acid sequences that bind to the same site to form a multivalent ligand may be useful to produce molecules that are capable of cross-linking together multiple receptor units. Multivalent ligands may also be constructed to combine amino acid sequences which bind to different sites.

a. Orientation of IRBAASs

In one aspect, the invention provides IRBPs that comprise two or more IRBAASs that are covalently linked at their N-termini or C-termini to form N-N, C-C, N-C, or C-N linked regions or peptides. These may be directed to the same IR site—Site 1-Site 1 or Site 2-Site 2 combinations. Alternatively, Site 1-Site 2 or Site 2-Site 1 combinations are provided. Site 2-Site 1 combinations are typically IR agonists. Such combinations can be referred to as “dimers.”

In specific embodiments, Site 1-Site 2 and Site 2-Site 1 orientations are possible. In addition, N-terminal to N-terminal (N-N); C-terminal to C-terminal (C-C); N-terminal to C-terminal (N-C); and C-terminal to N-terminal (C-N) linkages are possible. Accordingly, peptides may be oriented Site 1 to Site 2, or Site 2 to Site 1, and may be linked N-terminus to N-terminus, C-terminus to C-terminus, N-terminus to C-terminus, or C-terminus to N-terminus. In certain cases, a specific orientation may be preferable to others, for example, for maximal agonist or antagonist activity.

The orientation and linkage of the monomer subunits has been found to dramatically alter dimer activity. In particular, certain Site 1/Site 2 heterodimer sequences show agonist or antagonist activity at IR, depending on orientation and linkage of the constituent “monomer” “subunits” (IRBAASs). For example, a Site 1-Site 2 orientation (C-N linkage) shows antagonist activity at IR. In contrast, a Site 2-Site 1 orientation (C-N linkage) shows potent agonist activity at IR. Similarly, Site 1-Site 2 (C-N linkage) heterodimers show antagonist activity at IR, while Site 1-Site 2 (C-C or N-N linkage) heterodimers show agonist activity.

Whether produced by recombinant gene expression or by conventional chemical linkage technology, the various IRBAASs may be coupled through linkers of various lengths. In one aspect, IRBPs can be characterized by the inclusion of no linker or at most a very short linker between IRBAASs (e.g., a linker consisting of less than about 5 residues, such as 0, 1, or 2 residues). An intra-IRBAA “linker” typically consists of one or a few small and/or flexible typical amino acid residues (see Table 1 above), such as a Gly, a Val, and/or a Ser residue; one or more digestive enzymatic degradation-resistant unusual amino acid residues; one or more degradation-resistant non-amino acid moieties; or a combination of any thereof. Where one or more IRBAASs are linked to additional non-IRBAAS sequences (e.g., sequences that promote stabilization, targeting (such as a cholera toxin B fusion partner), detection (e.g., a green fluorescent protein (GFP) sequence, firefly luciferase sequence, epitope tag sequence, an enzyme substrate sequence; or similar sequence), stabilization (e.g., a ubiquitin sequence for improved production in E. coli or other stabilizing sequence), and/or purification (e.g., a hexa-histidine sequence or other His-tag; an epitope tag; or the like) of the IRBP or that impart additional pharmacological/biological functionality such as binding to a second target other than IR) in the context of a fusion protein, which also are provided by this invention, a linker between the IRBAAS(s) and the non-IRBAAS(s) may be significantly longer, particularly in the case of a fusion protein that comprises one or more secondary ligand-binding sequences/domains. Principles and techniques relevant to the selection and inclusion of such linker sequences are well known in the art. A specific example of such an IRBP fusion protein is embodied in IRBP S860, which comprises a His-tag and an ubiquitin fusion partner portion.

b. N-Terminal Acetylated/C-Terminal Amidated Multivalent IRBPs

In another aspect, the invention provides IRBPs that are characterized by N-terminal acylation, typically acetylation, of an included IRBAAS and/or C-terminal amidation of an included IRBAAS. For example, the invention in one aspect provides an IRBP that comprises one N-terminal and acetylated IRBAAS and a different C-terminal and amidated IRBAAS. The inventors have determined that such modifications surprisingly improve the modified molecule in terms of stability and/or IR binding. The IRBP in this context can comprise one or more IRBAAS as described herein (e.g., an IRBAA according to Formula 1a-1g) or a sequence (Formula) of one or more of the insulin-binding peptides described in the prior patent documents (e.g., a “Formula 4” sequence as described in US 20030236190). Typically, the N-terminal acetyl and/or C-terminal amide are directly linked to the termini of IRBAASs. However, in the case of addition variants/fusion proteins, these substituents can be associated with non-IRBAAS residues that are in turn directly or indirectly linked to “internal” IRBAASs.

In a particular aspect, the invention provides IRBPs comprising a sequence according to one or more of Formulas 6a-6g and either (a) a sequence according to one or more of Formulas 1 a-1g; (b) a sequence according to Formula 1as described in the prior patent documents; (c) a sequence according to Formula 2a; or (d) a sequence according to Formula 2 as described in the prior patent documents, wherein the IRBPs are characterized by N-terminal acetylation and/or C-terminal amidation. Typically, such IRBPs are “dimers” of two of such Formula 6/Formula 6-like (i.e., a sequence according to Formula 6 or Formulas 6a-6g) and non-Formula-6-like sequences (i.e., Formula 2, Formula 2a, or Formula 1a-1g sequence). Typically, such IRBPs are directly linked or separated by a very short linker (e.g., a linker of 1-3 residues or moieties). Typically, such dimers are oriented Site 2-Site 1 (C-N linkage).

The modification of other IRBPs provided in the prior patent documents by such N-terminal acetylation and/or C-terminal amidation modifications (e.g., a Formula 1-Formula 4 dimer) also is a feature of the invention. As the description of various Formula and sequences herein is to illustrate the novel and useful aspects of this invention, such IRBPs encompassed by the aspects are not specifically described herein, but are readily provided by the application of the inventive methods (e.g., the inclusion of both N-terminal acetylation and C-terminal amidation) to the IRBPs and IRBAASs included within the disclosure of the prior patent documents. The same principle applies to any aspect of the invention described here.

c. Degradation-Resistant Multivalent Derivatives

As described above, another important facet of the invention is multivalent IRBPs that include degradation-resistant amino acid residues and/or moieties.

In one aspect, the invention provides degradation-resistant IRBPs that comprise one or more Formula 6a-6g IRBAASs and at least one Formula 2 sequence of the prior patent documents (see, e.g., US 20030236190) or Formula 2a sequence described above, arranged in a Site 2-Site 1 orientation (C-N linkage). In a particular aspect, the invention provides IRBPs that comprise a single IRBAAS according to one or more of Formulas 6a-6g and a single Formula 2 or Formula 2a sequence comprising one or more degradation-resistant unusual amino acid (AA) residues and/or non-AA moieties located between the IRBAASs, at one or both termini of the IRBP, or both, wherein the sequence optionally is further characterized by inclusion of one or two linker residues between the respective IRBAASs, which may be in place of or in addition to one or more degradation-resistant moiety or residue linkers.

In another facet, the invention provides degradation-resistant IRBPs that comprise a Formula 6 IRBAAS and a Formula 2 IRBAAS as described in one or more of the prior patent documents, wherein the IRBP comprises a degradation resistant unusual residue or moiety between the IRBAASs or at either or both termini of the dimer. Such IRBPs typically have a Site 2-Site 1 orientation (C-N linkage).

In another aspect, the invention provides IRBPS that comprise at least one IRBAAS according to one or more of Formulas 1a-1g and at least one IRBAAS according to one or more of Formulas 6a-6g. In another aspect, the invention provides IRBPs that comprise at least one IRBAAS according to Formula 1 of the prior patent documents and at least one IRBAAS of Formulas 6a-6g. In still a further aspect, the invention provides IRBPS that comprise at least one Formula 6 sequence according to the prior patent documents and at least one sequence according to one or more of Formulas 1a-1g. In yet an additional aspect, the invention provides IRBPs according to any of the foregoing aspects of this paragraph, wherein the IRBP comprises one or more degradation-resistant unusual amino acid residues and/or degradation-resistant moieties located between the Formula 1 or Formula 1-like sequence (1a-1g sequence) and the Formula 6 or Formula 6-like (6a-6g) sequence, at one or both termini of the IRBP, or a combination thereof. In one aspect, the invention provides a “dimer” exhibiting one or more of the features described in this paragraph. Such IRBPs typically exhibit a Site 2-Site 1 orientation (C-N linkage).

In still another aspect, the invention provides an IRBP comprising a Formula 1 IRBAAS and a Formula 6 IRBAAS, which typically is a “dimer” thereof (and typically Site 2-Site 1 oriented (C-N linkage)) according to the prior patent documents (see, e.g., US 20030236190), wherein the IRBP comprises at least one degradation-resistant unusual amino acid residue and/or moiety between the IRBAASs and/or at one or both termini of the IRBP.

In a particular exemplary aspect, the invention provides IRBPs that comprise a Formula 6 or Formula 6-like sequence and a Formula 1 or Formula 1-like sequence, oriented and linked as described above, wherein one or more degradation resistant moieties or residues are present at positions 5, 7, and/or 8 of the Formula 6 or Formula 6-like sequence, one or two degradation-resistant moieties or residues are present at positions 1 or 2 of the Formula 1 or Formula 1-like sequence, or both. Such IRBPs also can further comprise N-terminal and/or C-terminal blocking groups (e.g., acetyl and amide groups, respectively).

Any of the IRBPs described herein can comprise terminally and/or internally positioned acyl derivatives linked to the amino acid sequence backbone thereof (e.g., to a Formula 2, Formula 6, and/or Formula 1 sequence and/or to one or more non-IRBAAS sequences) that also may increase the stability of the peptides. An acyl derivative in this context can be, for example, a C₁₂-C₂₂ carboxylic or dicarboxylic acid substituent (each sub-range and member hereof representing an individual aspect)). Such IRBPs can exhibit, for example, enhanced albumin binding/association, which in turn imparts increased in vivo half-life, as compared to other IRBPs and/or insulins. Other forms of acylation of IRBPs also can be suitable (e.g., as mentioned elsewhere herein).

In a particular aspect, the invention provides IRBPs comprising a Formula 1-like IRBAAS and a Formula 6-like IRBAAS (typically characterized by Site 2-Site 1 orientation, C-N linkage) wherein the Xaa₇ position of the Formula 6-like sequence is substituted with a degradation-resistant residue or moiety (e.g., an Aib) and the Xaa₁ residue of the Formula 1-like sequence is substituted with a degradation-resistant residue or moiety (e.g., a Dip). An example of such an IRBP is embodied in IRBP S873.

In a more general aspect, the invention provides IRBPs comprising a Formula 1-like IRBAAS and a Formula 6-like IRBAAS (typically characterized by Site 2-Site 1 orientation, C-N linkage), wherein the Xaa₅, Xaa₇, and/or Xaa₈ position of the Formula 6-like sequence is/are substituted with a degradation-resistant residue or moiety and the Xaa₁ and/or Xaa₅ residue(s) of the Formula 1-like sequence is/are substituted with a degradation-resistant residue or moiety.

With respect to any of the foregoing, the reference to a degradation-resistant residue or moiety at the termini of the IRBP should be understood as typically referring to the region defined by at least two IRBAASs; although in cases of variants that are modified by additions at one or both termini, such degradation-resistant residues/moieties may be associated with residues “outside” the context of the external IRBAASs themselves.

An unusual degradation-resistant amino acid residue or degradation-resistant moiety can be any suitable type of such a residue or moiety. Examples of unusual degradation-resistant residues include sarcosine, diphenylalanine, aminoisobutyric acid, D-arginine, and N-methyl-phenylalanine. Additional examples of such residues and degradation resistant moieties suitable for inclusion in multivalent IRBPs are described elsewhere herein.

In another exemplary aspect, the invention provides an IRBPS comprising at least two different IRBAASs according to different formulas (as provided herein and/or in the prior patent documents), which can be characterized by inclusion of at least two degradation-resistant amino acid residues or moieties positioned and selected such that that IRBP is more degradation-resistant than a similar sequence lacking the residues/moieties.

In a further aspect, the invention provides IRBPS according to any of the aspects described in this Section A.5.c., wherein the IRBP also can be characterized by the presence of N-terminal acetylation and/or C-terminal amidation, typically of IRBAAS residues of the various Formulas included therein.

The inclusion of similar degradation-resistant residues or moieties can be applied to other IRBPs described in the prior patent documents (such as Formula 1-Formula 4 dimers) in a similar manner. Such IRBPs also are a feature of the invention.

d. Exemplary Multivalent IRBPs

A number of new multivalent IRBPs have been developed in the context of this invention. Such multivalent IRBPs typically can be characterized by one or more of the foregoing classifications and/or that correspond to other IRBPs based on other criteria (e.g., based on the existence of a consensus sequence or motif found in these sequences not specifically disclosed here).

The following table presents a number of such exemplary and illustrative sequences:

TABLE 2

| | Sequence | | | | |
|---|---|---|---|---|---|
| | (see note below table for ID of unnatural | | | | rel. |
| [symbol] ef. No. | amino acids and linkers) | [symbol] d (HIR) | [symbol] el. to HI¹ | EC50 (FFC) | to HI¹ |
| 612 | HQLEEEWQAIQCELWGRGCPSESFYDWFERQL | [symbol] .5 * 10⁻¹² | [symbol] 6% | | |
| 613 | HLEEEWSEIQCELWGRGCPSESFYDWFERQL | [symbol] .3 * 10⁻¹² | [symbol] 8% | | |
| 616 | HQLEEEWQAIQCELWGRGCPSEDFYDWFEAQ | [symbol] .0 * 10⁻¹² | [symbol] 00% | 1.5 * 10⁻⁹ | 4% |
| | LHA | | | | |
| 617 | HLEEEWSEIQCELWGRGCPSEDFYDWFEAQLHA | [symbol] .7 * 10⁻¹² | [symbol] 4% | 3.9 * 10⁻⁹ | 1% |
| 618 | HELEEEWKRIECELWGRGCPSEDFYDWFEAQL | [symbol] .5 * 10⁻¹² | [symbol] 4% | 4.6 * 10⁻⁹ | 1% |
| | HA | | | | |
| 619 | Ac- | [symbol] .6 * 10⁻¹² | [symbol] 25% | | |
| | HQLEEEWQAIQCELWGRGCPSEDFYDWFEAQLHA | | | | |
| 620 | Ac- | [symbol] .8 * 10⁻¹² | [symbol] 11% | 6.9 * 10⁻⁸ | 1% |
| | HLEEEWSEIQCELWGRGCPSEDFYDWFEAQLHA | | | | |
| 621 | Ac- | [symbol] .2 * 10⁻¹² | [symbol] 1% | | |
| | HELEEEWKRIECELWGRGCPSEDFYDWFEAQLHA | | | | |
| 626 | SLEEEWAQIECEVYGRGCPSVRGFQGGTVWP | [symbol] .3 * 10⁻¹² | [symbol] 1% | 9.7 * 10⁻⁹ | 0.4% |
| | GYEWLRNAAKK | | | | |
| 634 | Ac- | [symbol] .6 * 10⁻¹² | [symbol] 25% | 8.3 * 10⁻¹¹ | 66% |
| | SLEEEWAQIQCEVWGRGCPSESFYDWFEAQLHA | | | | |
| 635 | Ac- | [symbol] .2 * 10⁻¹² | [symbol] 1% | | |
| | SLEEEWAQIQCEVWGRGCPSEDFYDWFEEQLHN | | | | |
| 636 | Ac- | [symbol] .6 * 10⁻¹² | [symbol] 25% | 5.6 * 10⁻¹¹ | 98% |
| | SLEEEWAQIQCEVWGRGCPSESFYDWFERQL | | | | |
| 637 | Ac-SLEEEWAQIECEVYGRGCPSEGFYNAIELLS | [symbol] .6 * 10⁻¹² | [symbol] 0% | 3.9 * 10⁻⁸ | 0.1% |
| 639 | Ac- | [symbol] .3 * 10⁻¹² | [symbol] 7% | 7.8 * 10⁻¹⁰ | 5% |
| | HGLEEEWAQIQCEVWGRGCPSESFYDWFEAQLHA | | | | |
| 640 | Ac- | [symbol] .4 * 10⁻¹¹ | [symbol] 4% | | |
| | SLEEEWAQIQCEVWGRGCPSESFYDWFERQLY | | | | |
| 641 | Ac- | [symbol] .5 * 10⁻¹² | [symbol] 42% | | |
| | SLEEEWAQIQAEVWGRGAPSESFYDWFEAQLHA | | | | |
| 642 | Ac- | [symbol] .7 * 10⁻¹² | [symbol] 5% | 1.4 * 10⁻¹⁰ | 39% |
| | SLEEEWAQIQCEVWGRGCQRPEPFYDWFERQL | | | | |
| 643 | Ac- | [symbol] .5 * 10⁻¹² | [symbol] 4% | 9.7 * 10⁻¹¹ | 57% |
| | SLEEEWAQIQCELWGRGCPSESFYDWFERQL | | | | |
| 645 | Ac- | [symbol] .6 * 10⁻⁹ | [symbol] .07% | | |
| | HGLEEEWAQHEEDVYHPPAESFYDWFEAQLHA | | | | |
| 648 | Ac- | [symbol] .0 * 10⁻¹² | [symbol] 5% | | |
| | SLEEEWAQIQCEVWGRGCPSEAFYDWFAEQLDD | | | | |
| 649 | SLEEEWAQIEAEVWGRGAPPSESFYDWFERQL | [symbol] .5 * 10⁻¹² | [symbol] 7% | 1.4 * 10⁻⁸ | 0.4% |
| | GY | | | | |
| 651 | Ac-SLEEEWAQIECEVYGRGC-pox- | [symbol] .2 * 10⁻¹² | [symbol] 08% | | |
| | FYDWFERQL | | | | |
| 653 | Ac- | [symbol] .7 * 10⁻¹² | [symbol] 4% | | |
| | SLEEEWAQIQCEVWGRGCPSEGFYDWFLQDDHV | | | | |
| 654 | Ac- | [symbol] .7 * 10⁻¹² | [symbol] 5% | | |
| | SLEEEWAQIQCEVWGRGCPSEVFYDWFYFDDHD | | | | |
| 655 | Ac- | [symbol] .0 * 10⁻¹² | [symbol] 0% | 6.3 * 10⁻¹⁰ | 8.7% |
| | SLEEEWAQIQCEVWGRGCQRPEPFYDWFAEQLDD | | | | |
| 657 | Ac- | [symbol] .1 * 10⁻¹¹ | [symbol] 09% | | |
| | SLEEEWAQIQCEVWGRGCKSESFYDWFERQL | | | | |
| 658 | Ac- | [symbol] .4 * 10⁻¹¹ | [symbol] .9% | antag. | |
| | SLEEEWAQIQCEVWGRGCPSGGSTFEDYLHNVVFVPRPS | | | (~1e−6) | |
| 659 | HOOC-C19- | [symbol] .3 * 10⁻¹⁰ | [symbol] 6% | | |
| | SLEEEWAQIECEVYGRGCPSESFYDWFERQL | | | | |
| 660 | MeO-PEG5000- | [symbol] .8 * 10⁻¹¹ | [symbol] 2% | 2.0 * 10⁻⁹ | 3.3% |
| | SLEEEWAQIECEVYGRGCPSESFYDWFERQL | | | | |
| 662 | Ac- | [symbol] .2 * 10⁻¹² | [symbol] 3% | 2.0 * 10⁻⁹ | 3.3% |
| | SLEEEWAQIQCEVWGRGCPSEAFYDWFHEQLDD | | | | |
| 663 | Ac-SLE-Aib-EW-Aib- | [symbol] .6 * 10⁻¹² | [symbol] 0% | 4.3 * 10⁻¹⁰ | 15% |
| | QIQCEVWGRGCPSESFYDWFERQL | | | | |
| 665 | Ac- | [symbol] .4 * 10⁻¹² | [symbol] 1% | 1.9 * 10⁻⁹ | 3.4% |
| | SLEEEWAQIECEVYGRGCPSESFYHWFERQL | | | | |
| 667 | Ac- | [symbol] .8 * 10⁻¹³ | [symbol] 86% | 2.1 * 10⁻¹⁰ | 31% |
| | SLEEEWAQIQCEVWGRGCPSESFYGWFERQL | | | | |
| 668 | Ac- | [symbol] .2 * 10⁻¹² | [symbol] 3% | 2.3 * 10⁻⁹ | 2.8% |
| | SLEEEWAQIQCEVWGRGCPSESFYGWFQAQL | | | | |
| 669 | GSSHHHHHHSSGLVPRGSHMQIFVKTLTGKTIT | [symbol] .7 * 10⁻¹¹ | [symbol] 8% | 1.7 * 10⁻⁹ | 3% |
| | LEVEPSDTIENVKAKIQDKEGIPPDQQRLIFAGKQLEDGRTL | | | | |
| | SDYNIQKESTLHLVLRLRGGIDKSLEEEWAQIECEVYGRGC | | | | |
| | PSESFYDWFERQL | | | | |
| 670 | GSLDESFYDWFERQLGKKY | [symbol] .5 * 10⁻⁸ | | 8.5 * 10⁻⁷ | |
| 671 | Ac- | [symbol] .7 * 10⁻¹² | [symbol] 4% | 1.0 * 10⁻¹⁰ | 65% |
| | SLEEEWAQIQCEVWGRGCPSESFYDWFERQLG | | | | |
| 672 | Ac- | [symbol] .4 * 10⁻¹¹ | [symbol] 7% | 2.5 * 10⁻⁸ | 0.3% |
| | SLEEEWAQIQAEVWGRGAPSESFYDWFERQL | | | | |
| 673 | Ac- | [symbol] .1 * 10⁻¹² | [symbol] 4% | | |
| | SLEEEWAQIQCEVWGRGCPSEGFYNAIELLS | | | | |
| 674 | Ac- | [symbol] .3 * 10⁻¹¹ | [symbol] 5% | 2.3 * 10⁻⁷ | |
| | SLEHEWAQIHCEVYGRGCPSESFYHWFERQL | | | | |
| 675 | GSLDESFYDWFERQLG-(pox)₂- | [symbol] .4 * 10⁻¹² | [symbol] 9% | 2.6 * 10⁻¹⁰ | 25% |
| | SLEEEWAQIQCEVWGRGCPSY | | | | |
| 677 | VQDDCRGRPCGDADSFYEWFDQQAM-(pox)₂- | [symbol] .4 * 10⁻¹² | [symbol] 1% | | |
| | SLEEEWAQIQCEVWGRGCP | | | | |
| 679 | Ac-SLEEEWAQIQCEVWGRGC-pox- | [symbol] .5 * 10⁻¹² | [symbol] 3% | | |
| | GFYGWFNAQLA | | | | |
| 680 | SLEEHWAQVECEVYGRGCPSESFYDWFERQL | [symbol] .8 * 10⁻¹¹ | [symbol] 3% | 1.6 * 10⁻⁸ | 0.4% |
| 681 | SLEEEWGQVECEVYGRGCPSESFYDWFERQL | [symbol] .9 * 10⁻¹¹ | [symbol] 6% | 1.3 * 10⁻⁸ | 0.4% |
| 682 | SLEEEWAQVECEVYGRCPSESFYDWFERQL | [symbol] .1 * 10⁻¹¹ | [symbol] 9% | 7.7 * 10⁻⁹ | 0.8% |
| 683 | SLEEEWAQVECEVYGRNCPSESFYDWFERQL | [symbol] .5 * 10⁻¹¹ | [symbol] 8% | 2.1 * 10⁻⁹ | 2.9% |
| 684 | SLEEEWAQVECEVYQRGCPSESFYDWFERQL | [symbol] .3 * 10⁻¹¹ | [symbol] 0% | 1.5 * 10⁻⁸ | 0.4% |
| 686 | SLEEEWAQKECEVYGRGCPSESFYDWFERQL | [symbol] .3 * 10⁻⁹ | [symbol] .5% | | |
| 687 | SLEEEWAQRECEVYGRGCPSESFYDWFERQL | [symbol] .9 * 10⁻⁹ | [symbol] .3% | | |
| 688 | SLEEEWAQHECEVYGRGCPSESFYDWFERQL | [symbol] .6 * 10⁻¹⁰ | [symbol] .5% | 6.8 * 10⁻⁷ | |
| 689 | SLEEYWAQVECEVYGRGCPSESFYDWFERQL | [symbol] .2 * 10⁻¹¹ | [symbol] 7% | 1.5 * 10⁻⁸ | 0.4% |
| 690 | SLEEVWAQVECEVYGRGCPSESFYDWFERQL | [symbol] .3 * 10⁻¹¹ | [symbol] 3% | 1.6 * 10⁻⁸ | 0.4% |
| 691 | SLEEEWYQVECEVYGRGCPSESFYDWFERQL | [symbol] .1 * 10⁻¹¹ | [symbol] 1% | 2.5 * 10⁻⁸ | 0.3% |
| 692 | SLEEEWAQVECEVYGRHCPSESFYDWFERQL | [symbol] .1 * 10⁻¹² | [symbol] 0% | 7.7 * 10⁻⁹ | 0.8% |
| 693 | SLEEEWAQVECEVYGRVCPSESFYDWFERQL | [symbol] .1 * 10⁻¹¹ | [symbol] 9% | 7.5 * 10⁻⁸ | 0.1% |
| 694 | SLEEEWAQVECEVYGRFCPSESFYDWFERQL | [symbol] .0 * 10⁻¹¹ | [symbol] 1% | 2.6 * 10⁻⁸ | 0.3% |
| 695 | SLEEEWAQVECEVYHRGCPSESFYDWFERQL | [symbol] .1 * 10⁻¹¹ | [symbol] 4% | 9.8 * 10⁻⁹ | 0.6% |
| 696 | SLEEEWAQVECEVYERGCPSESFYDWFERQL | [symbol] .9 * 10⁻¹¹ | [symbol] 4% | 8.7 * 10⁻⁷ | |
| 697 | SLEEHWHQVECEVYGRGCPSESFYDWFERQL | [symbol] .0 * 10⁻¹⁰ | [symbol] .5% | 7.2 * 10⁻⁸ | 0.1% |
| 698 | SLEEHWHQVECEVYHRHCPSESFYDWFERQL | [symbol] .0 * 10⁻¹⁰ | [symbol] % | 1.9 * 10⁻⁷ | 0.03% |
| 699 | Ac- | [symbol] .4 * 10⁻¹² | [symbol] 8% | 4.3 * 10⁻⁹ | 1.5% |
| | SLEEEWAQVECEVYGRGCPSESFYDWFERQL | | | | |
| 700 | Ac- | [symbol] .0 * 10⁻¹² | [symbol] 3% | | |
| | SLEEEWAQIQEEVWGRGEPSESFYDWFERQL | | | | |
| 713 | Carboxyfluorescein-pox- | [symbol] .1 * 10⁻¹⁰ | [symbol] % | | |
| | SLEEEWAQIECEVYGRGCPSESFYDWFERQL | | | | |
| 714 | Biotin-pox- | [symbol] .1 * 10⁻¹² | [symbol] 1% | | |
| | SLEEEWAQIECEVYGRGCPSESFYDWFERQL | | | | |
| 722 | HOOC-C19- | [symbol] .9 * 10⁻¹⁰ | [symbol] 0% | | |
| | (pox)₄SLEEEWAQIQCEVWGRGCPSESFYDWFERQL | | | | |
| 726 | Ac-SLEEEWAQIECEVWGRGCPSEGFYNAIELLS | [symbol] .2 * 10⁻¹¹ | [symbol] 00% | 1.9 * 10⁻⁸ | 0.3% |
| 727 | Ac- | [symbol] .8 * 10⁻¹¹ | [symbol] 7% | | |
| | SLEEEWAQIEAEVWGRGAPSESFYDWFERQLG | | | | |
| 728 | GSLDESFYDWFERQLG-pox-pox- | [symbol] .9 * 10⁻¹¹ | [symbol] 3% | partial | |
| | SLEEEWAQIQCEVWGRGCPSESFYDWFERQL | | | | |
| 729 | YEEESFYDWFERQLGEE | [symbol] .1 * 10⁻⁸ | | | |
| 731 | Ac-SLEEEWAQIQCEVWGRGCPS | [symbol] .3 * 10⁻¹⁰ | [symbol] .5% | | |
| 732 | YEEESFYGWFERQLGEE | [symbol] .8 * 10⁻⁹ | [symbol] .1% | >2 * 10⁻⁶ | |
| 733 | Ac- | [symbol] .1 * 10⁻¹² | [symbol] 61% | 1.2 * 10⁻¹⁰ | 61% |
| | SLEEEWAQIQCEVWGRGCPSESFYGWFERQLG | | | | |
| 734 | Ac- | [symbol] .6 * 10⁻¹² | [symbol] 78% | | |
| | SLEEEWAQVECEVWGRHCPSESFYGWFERQLG | | | | |
| 739 | SLEEEWAQIQCEVWGRGCPSESFYDWFERQLK | [symbol] .1 * 10⁻¹¹ | [symbol] .5% | | |
| | KKKKK | | | | |
| 740 | KKKKKKSLEEEWAQIQCEVWGRGCPSESFYDW | [symbol] .6 * 10⁻¹¹ | [symbol] .3% | | |
| | FERQL | | | | |
| 753 | EEESFYDWFERQLGEEY | [symbol] .8 * 10⁻⁸ | | | |
| 764 | Ac- | [symbol] .9 * 10⁻¹¹ | [symbol] 7% | | |
| | SLEEEWAQIQCEVWGRGCPSQILKELEESSFRKTFEDYLH | | | | |
| | NVVFVPRPS | | | | |
| 767 | Ac- | [symbol] .5 * 10⁻¹² | [symbol] 20% | | |
| | SLEEEWAQIQCEVWGRPCPSESFYGWFERQLG | | | | |
| 768 | Ac- | [symbol] .6 * 10⁻¹² | [symbol] 38% | | |
| | SLEEEWAQIQCEVWGRGCPSESFYGWFEAQLG | | | | |
| 769 | Ac- | [symbol] .7 * 10⁻¹² | [symbol] 00% | | |
| | SLEEEWAQIQCEVWGRGCPSESFYDWFERQLK | | | | |
| 773 | C14- | [symbol] .1 * 10⁻¹⁰ | [symbol] .1% | | |
| | SLEEEWAQIQCEVWGRGCPSESFYDWFERQL | | | | |
| 774 | Ac-SLEEEWAQIQCEVWGRGCPSESFY-Sar- | [symbol] .8 * 10⁻¹⁰ | [symbol] 2% | | |
| | WFERQLG | | | | |
| 775 | Ac- | [symbol] .7 * 10⁻¹² | [symbol] 64% | | |
| | SLEEEWAQIQCEVWGRGCPSESFYGWFERQL-Sar | | | | |
| 776 | Ac-SLEEEWAQIQCEVWGR-Sar- | [symbol] .5 * 10⁻¹¹ | [symbol] 5% | | |
| | CPSESFYGWFERQL-Sar | | | | |
| 777 | Ac-SLEEEWAQIQCEVW-Sar- | [symbol] .9 * 10⁻¹¹ | [symbol] 1% | | |
| | RGCPSESFYGWFERQL-Sar | | | | |
| 778 | Ac-SLEEEWAQIQCEVW-Sar-R-Sar- | [symbol] .0 * 10⁻¹⁰ | [symbol] .9% | | |
| | CPSESFYGWFERQL-Sar | | | | |
| 779 | Ac-SLEEEWAQIQCEVW-Sar-R-Sar-CPSESFY- | [symbol] .7 * 10⁻⁸ | [symbol] .05% | | |
| | Sar-WFERQL-Sar | | | | |
| 788 | Ac-SLEEEWAQIQCEVWGRGCPSES-Tbp- | [symbol] .1 * 10⁻⁹ | [symbol] .4% | | |
| | YGWFERQLG | | | | |
| 789 | Ac-SLEEEWAQIQCEVWGRGCPSES-Pya2- | [symbol] .8 * 10⁻¹⁰ | [symbol] 6% | | |
| | YGWFERQLG | | | | |
| 790 | Ac-SLEEEWAQIQCEVWGRGCPSES-Phg- | [symbol] .0 * 10⁻¹⁰ | [symbol] 4% | | |
| | YGWFERQLG | | | | |
| 791 | Ac-SLEEEWAQIQCEVWGRGCPSES-Hph- | [symbol] .4 * 10⁻¹¹ | [symbol] 4% | | |
| | YGWFERQLG | | | | |
| 792 | Ac-SLEEEWAQIQCEVWGRGCPSES-Dip- | [symbol] .6 * 10⁻¹¹ | [symbol] 1% | | |
| | YGWFERQLG | | | | |
| 793 | Ac-SLEEEWAQIQCEVWGRGCPSES-Cha- | [symbol] .4 * 10⁻¹¹ | [symbol] 5% | | |
| | YGWFERQLG | | | | |
| 794 | Ac-SLEEEWAQIQCEVWGRGCPSES-Bip- | [symbol] .5 * 10⁻¹⁰ | [symbol] 2% | | |
| | YGWFERQLG | | | | |
| 795 | Ac-SLEEEWAQIQCEVWGRGCPSES-Aic- | [symbol] .9 * 10⁻¹⁰ | [symbol] .4% | | |
| | YGWFERQLG | | | | |
| 799 | Ac- | [symbol] .8 * 10⁻¹¹ | 4% | | |
| | SLEHEWAQIQCEVWGRGCPSEPFYGWFLAQLG | | | | |
| 800 | Ac- | [symbol] .7 * 10⁻¹¹ | 07% | | |
| | SLEHEWAQIQCEVWGRGCPSEPFYGWFERQLG | | | | |
| 801 | Ac- | [symbol] .9 * 10⁻¹⁰ | [symbol] .5% | | |
| | SLEHEWAQIHCEVWGRGCPSESFYHWFERQLG | | | | |
| 802 | Ac-SLEEEWAQIQCEVWGRGC-pox- | [symbol] .4 * 10⁻¹¹ | 6% | | |
| | FYGWFERQLG | | | | |
| 805 | Ac-SLEEEWAQIQCEVWG-Orn- | [symbol] .3 * 10⁻¹¹ | 24% | | |
| | GCPSESFYGWFERQLG | | | | |
| 806 | Ac-SLE-Aib-EW-Aib-QIQCEVWGRGC-pox- | [symbol] .4 * 10⁻¹¹ | 3% | | |
| | FYGWFE-Aib-QLG | | | | |
| 808 | Ac-SLEHEWAQIQCEVWGRGCPSESFY-Sar- | [symbol] .2 * 10⁻⁹ | [symbol] .3% | | |
| | WFERQLG | | | | |
| 809 | Ac-SLEHEWAQIQCEVWGRGCPSESFY-Sar- | [symbol] .9 * 10⁻⁹ | [symbol] .7% | | |
| | WFHEQLG | | | | |
| 813 | Ac-SLEHEWAQIQCEVWGRPCPSEP(N- | [symbol] .1 * 10⁻⁹ | [symbol] .0% | | |
| | MePhe)Y-Sar-W(N-MePhe)HEQL-Sar | | | | |
| 860 | GSSHHHHHHSSGLVPRGSHMQIFVKTLTGKTIT | [symbol] .6 * 10⁻¹¹ | 3% | | |
| | LEVEPSDTIENVKAKIQDKEGIPPDQQRLIFAGKQLEDGRTL | | | | |
| | SDYNIQKESTLHLVLRLRGGIDKSLEEEWAQIQCEVWGRG | | | | |
| | CPSESFYDWFERQL | | | | |
| 861 | Ac-SLEHEWAQIQCEVWGRPCPSEPFY-Sar- | [symbol] 1 * 10⁻⁸ | | | |
| | WFHEQL-Sar | | | | |
| 862 | Ac-SLEHEWAQIQCEVWGRPCPSEP(N- | [symbol] 1 * 10⁻⁸ | | | |
| | MePhe)Y-Sar-WFHEQL-Sar | | | | |
| 863 | Ac-SLEHEWAQIQCEVWGRPCPSEPFY-Sar- | [symbol] 1 * 10⁻⁸ | | | |
| | W(N-MePhe)HEQL-Sar | | | | |
| 864 | Ac-SLEHEW-Aib-QIQCEVWGRPCPSEP-Dip-Y- | [symbol] .3 * 10⁻⁹ | | | |
| | Sar-WFHEQLGPP | | | | |
| 865 | Ac-SLEHEW-Aib-QIQCEVWGRPCrrrrrrrrrrPFY- | [symbol] 1 * 10⁻⁸ | | | |
| | Sar-WFHEQLGPP | | | | |
| 869 | Ac-rrrrrrrrrrrr-atan- | [symbol] 1 * 10⁻⁸ | | | |
| | SLEHEWAQIQCEVWGRPCPSEPFY-Sar-WFHEQL-Sar | | | | |
| 870 | Ac-SLEEEWAQIQCEVWGRGCPK(ε- | [symbol] .1 * 10⁻¹¹ | 7% | | |
| | cholate)ESFYGWFERQLG | | | | |
| 871 | Ac-SLEEEWAQIQCEVWGRGCPK(ε- | [symbol] .4 * 10⁻¹¹ | 4% | | |
| | carboxyfluorescein)ESFYGWFERQLG | | | | |
| 872 | Ac-SLEHEW-Aib-QIQCEVWGRPCPSEP-Dip- | [symbol] .1 * 10⁻¹⁰ | 6% | | |
| | YGWFHEQLGPP | | | | |
| 873 | Ac-SLEEEW-Aib-QIQCEVWGRPCPSEP-Dip- | [symbol] .9 * 10⁻¹¹ | 20% | | |
| | YGWFHEQLGPP | | | | |
| 874 | Ac-SLE-Aib-EW-Aib-QIQCEVWGRPCPSEP-Dip- | [symbol] .2 * 10⁻¹¹ | 8% | | |
| | YGWFHEQLGPP | | | | |
| 875 | Ac-SLEEEW-Aib- | [symbol] .5 * 10⁻¹¹ | 12% | | |
| | QIQCEVWGRGCPSESFYDWFERQL | | | | |
| 876 | Ac-SLEEEWA-Aib- | [symbol] .4 * 10⁻¹¹ | [symbol] 16% | | |
| | IQCEVWGRGCPSESFYDWFERQL | | | | |
| 877 | Ac-SLEEEWAQIQCEVWGRGCPSESFY-Aib- | [symbol] .9 * 10⁻¹¹ | [symbol] 7% | | |
| | WFERQL | | | | |
| 878 | Ac-SLEEEWAQIQCEVWGRGCPSESFYDWF- | [symbol] .6 * 10⁻¹¹ | [symbol] 02% | | |
| | Aib-RQL | | | | |
| 879 | Ac- | | | | |
| | SLEEEWAPIQCEVWGRGCPSESFYDWFERQL | | | | |
| 880 | Ac- | [symbol] .1 * 10⁻¹⁰ | [symbol] 5% | | |
| | SLEEEWAQIQCEVWGRGCPSESFYDWFPRQL | | | | |
| 881 | Ac- | [symbol] .4 * 10⁻¹¹ | [symbol] 5% | | |
| | SLEEEWAQIQCEVWGRGCPSESFYPWFERQL | | | | |
| 882 | Ac-SLEHEW-Aib-QIQCEVWGRPCPSEP-N- | [symbol] .7 * 10⁻¹⁰ | [symbol] .4% | | |
| | MePhe)-YGWFHEQLG | | | | |
| 883 | Ac-SLEHEW-Aib-QIQCEVWGRPCPSEPFYGW- | [symbol] .3 * 10⁻⁹ | [symbol] .7% | | |
| | (N-MePhe)-HEQLG | | | | |
| 884 | Ac- | [symbol] .2 * 10⁻¹¹ | [symbol] 41% | | |
| | SLEEEWAKIQCEVWGRGCPSESFYDWFERQL | | | | |
| 885 | Ac-SLEEEWAQIQCEVWGRGCPK(ε-atan-C19- | | | | |
| | COOH)ESFYGWFERQLG | | | | |
| 886 | Ac-SLEEEWAQIQCEVWGRGCPK(ε-atan-atan- | | | | |
| | C19-COOH)ESFYGWFERQLG | | | | |
| 887 | Ac-SLEHEW-Aib-QIQCEVWGRPCPK(ε-dde)EP- | | | | |
| | Dip-YGWFHEQLG | | | | |
| 888 | Ac-SLEEEWAQIQCEVWGRGCPK(ε- | | | | |
| | Ac)ESFYGWFERQLG | | | | |
| 889 | Ac-SLEHEW-Aib-QIQCEVWGRPCPK(ε- | [symbol] .0 * 10⁻¹¹ | [symbol] 7% | | |
| | cholate)EP-Dip-YGWFHEQLG | | | | |
| 890 | Ac-SLEHEW-Aib-QIQCEVWGRPCPK(ε-C19- | | | | |
| | COOH)EP-Dip-YGWFHEQLG | | | | |
| 892 | Ac-SLEEEW-Aib-QIQCEVWGRPCPSEP-Dip- | | | | |
| | YGW-Dip-HEQLGPP | | | | |
| 893 | Ac-SLEEEW-Aib-QIQCEVWGRPCPSEP-Dip- | | | | |
| | YGWF-Aib-EQLGPP | | | | |

¹Kd/EC50 value expressed relative to that of human insulin in the same assay (to account for assay-to-assay variability) [symbol]
indicates data missing or illegible when filed

Abbreviations
Sar sarcosine=N-methylglycine
Aib aminoisobutyric acid
Dip diphenylalanine
N-MePhe N-methyl-phenylalanine
r D-arginine
Orn ornithine
Tbp 4-tertbutyl-phenylalanine
Pya2 pyridylalanine
Phg phenylglycine
Hph homophenylalanine
Cha cyclohexylalanine
Bip 4-biphenylalanine
Aic 2-aminoindane-2-carboxylic acid
pox N-Fmoc-8-amino-3,6-dioxaoctanoic acid
a tan N-Fmoc-19-amino-5-oxo-3,10,13,16-tetraoxa-6-aza-nonadecanoic acid
Ac acetyl
C14 C14-monocarboxylic acid
HOOC—C19 C20-dicarboxylic acid
/C19—COON
MeO—PEG5000 polyethylene glycol, MW=5000
ε-dde 1-(4,4-dimethyl-2,6-dioxocyclohexylidene)ethyl

Each of these peptides, variants thereof, derivatives of either thereof, nucleic acid sequences encoding such peptides, and the use of such nucleic acids, peptides, variants, and/or derivatives, represent additional illustrative aspects of this invention.

As indicated in Table 2, a number of these exemplary IRBPs have been analyzed for human IR affinity (as shown by Kd and, in the adjacent column in terms normalized to the affinity of human insulin for the human IR on the same day—so as to reduce/eliminate assay-to-assay variations) and/or for effect on glucose uptake in mouse adipocytes (as illustrated in terms of EC50 (FFC)—which is the concentration needed for half maximal uptake of glucose in mouse adipocytes; note the column adjacent right to this data reflects data normalized for the action of human insulin on the same day in the same assay), as obtained by methods described in detail in the prior patent documents.

These data reflect the efficacy of novel IRBPs provided by this invention in terms of IR binding and also in terms of IR activity modulation (particularly agonism).

6. Additional IRBP Derivatives

As described above, the invention provides IRBP derivatives which specifically include the enzyme degradation-resistant derivates, acetylated/amidated derivatives, and other derivatives specifically described elsewhere herein.

The term derivative generally refers to a protein in which one or more of the amino acid residues of the peptide have been chemically modified (e.g., by alkylation, acylation, ester formation, amide formation, or other similar type of modification) or covalently associated with one or more heterologous substituents (e.g., a lipophilic substituent, a PEG moiety, a peptide side chain linked by a suitable organic moiety linker, etc.). The second type of derivative can separately be described as a conjugate. Because derivatives can vary significantly from their “naked” protein counterparts, they often can be considered unique aspects of the invention.

In general, IRBPs described herein can be modified by inclusion of any suitable number of such modified amino acids and/or associations with such conjugated substituents. Suitability in this context general is determined by the ability to at least substantially retain IR selectivity and/or specificity associated with the non-derivatized parent IRBP/IRBAAS. The inclusion of one or more modified amino acids may be advantageous in, for example, (a) increasing polypeptide serum half-life, (b) reducing polypeptide antigenicity, or (c) increasing polypeptide storage stability. Amino acid(s) are modified, for example, co-translationally or post-translationally during recombinant production (e.g., N-linked glycosylation at N-X-S/T motifs during expression in mammalian cells) or modified by synthetic means. Non-limiting examples of a modified amino acid include a glycosylated amino acid, a sulfated amino acid, a prenlyated (e.g., farnesylated, geranylgeranylated) amino acid, an acetylated amino acid, an acylated amino acid, a PEGylated amino acid, a biotinylated amino acid, a carboxylated amino acid, a phosphorylated amino acid, and the like. References adequate to guide one of skill in the modification of amino acids are replete throughout the literature. Exemplary protocols are found in, e.g., Walker (1998) PROTEIN PROTOCOLS ON CD-ROM Humana Press, Towata, N.J. Typically, the modified amino acid is selected from a glycosylated amino acid, a PEGylated amino acid, a farnesylated amino acid, an acetylated amino acid, a biotinylated amino acid, an amino acid conjugated to a lipid moiety, and an amino acid conjugated to an organic derivatizing agent.

IRBPs can be chemically modified by covalent conjugation to a polymer to increase their circulating half-life, for example. Exemplary polymers and methods to attach such polymers to peptides are illustrated in, e.g., U.S. Pat. Nos. 4,766,106; 4,179,337; 4,495,285; and 4,609,546. Additional illustrative polymers include polyoxyethylated polyols and polyethylene glycol (PEG) moieties (e.g., a IRBP can be conjugated to a PEG with a molecular weight of between about 1,000 and about 40,000, such as between about 2000 and about 20,000, e.g., about 3,000-12,000, and even more particularly about 5,000).

Thus, the peptides of the invention may be subjected to one or more modifications known in the art, which may be useful for manipulating storage stability, pharmacokinetics, and/or any aspect of the bioactivity of the peptide, such as, e.g., potency, selectivity, and drug interaction. Chemical modification to which the peptides may be subjected includes, without limitation, the conjugation to a peptide of one or more of polyethylene glycol (PEG), monomethoxy-polyethylene glycol, dextran, poly-(N-vinyl pyrrolidone) polyethylene glycol, propylene glycol homopolymers, a polypropylene oxide/ethylene oxide co-polymer, polypropylene glycol, polyoxyethylated polyols (e.g., glycerol) and polyvinyl alcohol, colominic acids or other carbohydrate based polymers, polymers of amino acids, and biotin derivatives. PEG conjugation of proteins at Cys residues is disclosed, e.g., in Goodson, R. J. & Katre, N. V. (1990) Bio/Technology 8, 343 and Kogan, T. P. (1992) Synthetic Comm. 22, 2417.

Other useful modifications include, without limitation, acylation, particularly N-terminal acylation of an IRBAAS (e.g., an N-terminally located Formula 6 or Formula 6a-6g IRBAAS) as described above, which may be obtained, e.g., using methods and compositions such as described in, e.g., U.S. Pat. No. 6,251,856, and WO 00/55119.

B. IRBP-Encoding Nucleic Acids and Related Compositions

In another facet, the invention provides IRBP-encoding nucleic acids.

IRBP-encoding nucleic acids can have any suitable characteristics and comprise any suitable features. Thus, for example, a IRBP-encoding nucleic acid may be in the form of DNA, RNA, or a hybrid thereof, and may include nonnaturally-occurring bases or nucleotide analogues, replacement of sugar moieties, conjugation of additional molecules (e.g., uptake promoting molecules); inclusion of a modified backbone (e.g., a phosphothioate backbone that promotes stability of the nucleic acid), secondary structure-promoting sequences, or combinations of such features. A nucleic acid typically advantageously comprises features that promote desired expression, replication, and/or selection in target host cell(s). Examples of such features include an origin of replication component, a selection gene component, a promoter component, an enhancer element component, a polyadenylation sequence component, a termination component, and the like, numerous suitable examples of which are known.

In a further aspect, the invention provides a vector comprising an IRBP-encoding nucleic acid. A vector refers to a delivery vehicle that promotes the expression of a IRBP-encoding nucleic acid, the production of a IRBP peptide, the transfection/transformation of target cells, the replication of the IRBP-encoding nucleic acid, promotes stability of the nucleic acid, promotes detection of the nucleic acid and/or transformed/transfected cells, or otherwise imparts advantageous biological and/or physiochemical function to the IRBP-encoding nucleic acid. A vector in the context of this invention can be any suitable vector, including chromosomal, non-chromosomal, and synthetic nucleic acid vectors (a nucleic acid sequence comprising a suitable set of expression control elements). Examples of such vectors include derivatives of SV40, bacterial plasmids, phage DNA, baculovirus, yeast plasmids, vectors derived from combinations of plasmids and phage DNA, and viral nucleic acid (RNA or DNA) vectors. In one exemplary aspect, a IRBP-encoding nucleic acid is comprised in a naked DNA or RNA vector, including, for example, a linear expression element (as described in, e.g., Sykes and Johnston (1997) Nat Biotech 17: 355-59), a compacted nucleic acid vector (as described in, e.g., U.S. Pat. No. 6,077,835 and/or International Patent Application WO 00/70087), a plasmid vector such as pBR322, pUC 19/18, or pUC 118/119, a “midge” minimally-sized nucleic acid vector (as described in, e.g., Schakowski et al. (2001) Mol Ther 3: 793-800), or as a precipitated nucleic acid vector construct, such as a CaP0₄-precipitated construct (as described in, e.g., International Patent Application WO 00/46147, Benvenisty and Reshef (1986) Proc Natl Acad Sci USA 83: 9551-55, Wigler et al. (1978), Cell 14:725, and Coraro and Pearson (1981) Somatic Cell Genetics 7:603). Such nucleic acid vectors and the usage thereof are well known in the art (see, e.g., U.S. Pat. Nos. 5,589,466 and 5,973,972).

In one aspect, the vector is suitable for expression of the IRBP in a bacterial cell. Examples of such vectors include, for example, vectors which direct high level expression of fusion proteins that are readily purified (e.g., multifunctional E. coli cloning and expression vectors such as BLUESCRIPT (Stratagene), pIN vectors (Van Heeke & Schuster, J Biol Chem 264: 5503-5509 (1989); pET vectors (Novagen, Madison, Wis.); and the like).

An expression vector also or alternatively can be, for example, a vector suitable for expression in a yeast system. Any vector suitable for expression in a yeast system can be employed. Suitable vectors for use in, e.g., Saccharomyces cerevisiae include, for example, vectors comprising constitutive or inducible promoters such as alpha factor, alcohol oxidase and PGH (reviewed in, e.g., Ausubel, supra, and Grant et al., Methods in Enzymol 153: 516-544 (1987)).

A nucleic acid and/or vector can also comprise a nucleic acid sequence encoding a secretion/localization sequence, which can target a polypeptide, such as a nascent polypeptide chain, to a desired cellular compartment, membrane, or organelle, or which directs polypeptide secretion to periplasmic space or into cell culture media. Such sequences are known in the art, and include secretion leader or signal peptides, organelle targeting sequences (e.g., nuclear localization sequences, ER retention signals, mitochondrial transit sequences, chloroplast transit sequences), membrane localization/anchor sequences (e.g., stop transfer sequences, GPI anchor sequences), and the like.

IRBP-encoding nucleic acid vectors can comprise or be associated with any suitable promoter, enhancer, and other expression-facilitating elements. Examples of such elements include strong expression promoters (e.g., a human CMV IE promoter/enhancer, an RSV promoter, SV40 promoter, SL3-3 promoter, MMTV promoter, or HIV LTR promoter), effective poly (A) termination sequences, an origin of replication for plasmid product in E. coli, an antibiotic resistance gene as a selectable marker, and/or a convenient cloning site (e.g., a poly-linker). Nucleic acids also can comprise an inducible promoter as opposed to a constitutive promoter such as CMV IE (the skilled artisan will recognize that such terms are actually descriptors of a relative degree of gene expression under certain conditions). In one aspect, the invention provides a nucleic acid comprising a sequence encoding an IRBP which is operatively linked to a tissue specific promoter (e.g., a promoter specific for a tissue that the IRBP shows selectivity for on the basis of IR isoform specificity).

In another aspect, the nucleic acid is positioned in and/or delivered to the host cell or host animal via a viral vector. Any suitable viral vector can be used in this respect, and several are known in the art. A viral vector can comprise any number of viral polynucleotides, alone or in combination with one or more viral proteins, which facilitate delivery, replication, and/or expression of the nucleic acid of the invention in a desired host cell. The viral vector can be a polynucleotide comprising all or part of a viral genome, a viral protein/nucleic acid conjugate, a virus-like particle (VLP), a vector similar to those described in U.S. Pat. No. 5,849,586 and International Patent Application WO 97/04748, or an intact virus particle comprising viral nucleic acids and the nucleic acid of the invention. A viral particle viral vector can comprise a wild-type viral particle or a modified viral particle. The viral vector can be a vector which requires the presence of another vector or wild-type virus for replication and/or expression (i.e., a viral vector can be a helper-dependent virus), such as an adenoviral vector amplicon. Typically, such viral vectors consist essentially of a wild-type viral particle, or a viral particle modified in its protein and/or nucleic acid content to increase transgene capacity or aid in transfection and/or expression of the nucleic acid (examples of such vectors include the herpes virus/AAV amplicons). Typically, a viral vector is similar to and/or derived from a virus that normally infects humans. Suitable viral vector particles in this respect, include, for example, adenoviral vector particles (including any virus of or derived from a virus of the adenoviridae), adeno-associated viral vector particles (AAV vector particles) or other parvoviruses and parvoviral vector particles, papillomaviral vector particles, flaviviral vectors, alphaviral vectors, herpes viral vectors, pox virus vectors, retroviral vectors, including lentiviral vectors. Examples of such viruses and viral vectors are in, e.g., Fields et al., eds. , VIROLOGY Raven Press, Ltd., New York (3^{rd} ed., 1996 and 4^{th} ed., 2001); ENCYCLOPEDIA OF VIROLOGY, R. G. Webster et al., eds., Academic Press (2nd ed., 1999); FUNDAMENTAL VIROLOGY, Fields et al., eds., Lippincott-Raven (3rd ed., 1995), Levine, “Viruses,” Scientific American Library No. 37 (1992), MEDICAL VIROLOGY, D. O. White et al., eds., Acad. Press (2nd ed. 1994), and INTRODUCTION TO MODERN VIROLOGY, Dimock, N. J. et al., eds., Blackwell Scientific Publications, Ltd. (1994).

Viral vectors that can be employed with polynucleotides and other nucleic acids of the invention and the methods described herein thus include, for example, adenoviral viral vectors; adeno-associated viral (AAV) vectors; papillomaviral vectors; herpes viral vectors; retroviral vectors, including lentiviral vectors, pox viral vectors (e.g., vaccinia virus vectors); and the like.

The invention also provides recombinant cells, such as yeast, bacterial, and mammalian cells (e.g., immortalized mammalian cells) comprising an IRBP-encoding nucleic acid, vector, or combination of either or both thereof. For example, in one exemplary aspect the invention provides a cell comprising a nucleic acid stably integrated into the cellular genome that comprises a sequence coding for expression of an IRBP of the invention. In another aspect, the invention provides a cell comprising a non-integrated nucleic acid, such as a plasmid, cosmid, phagemid, or linear expression element, which comprises a sequence coding for expression of an IRBP.

Nucleic acids, vectors, and cells can be used as surrogates for IRBP proteins of the invention in most of the inventive methods described herein, e.g., so as to “deliver” one or more IRBPs to a cell in culture or a host. Thus, for example, the invention provides a method of modulating IR activity in a mammal that comprises introducing a vector comprising a IRBP-encoding nucleic acid to suitable cells under conditions suitable for expression of the nucleic acid and production of a IRBP such that the expressed IRBP comes in contact with cells comprising IRs responsive thereto so as to modulate IR activity therein.

The invention also provides transgenic organisms comprising IRBP-encoding nucleic acids or vectors comprising the same. Suitable transgenic organisms include mice, rats, chickens, plants, cows, goats, guinea pigs, monkeys, and other non-human primates. Transgenic animals can be produced by stable introduction of IRBP-encoding nucleic acids according to standard techniques.

C. IRBP Compositions

IRBPs can be provided in a homogenous composition or in combination with other active and/or inert ingredients (e.g., in one aspect, the invention provides a composition comprising one or more IRBPs according to the disclosure herein and one or more active secondary agents, such as one or more insulin secretagogues, alpha-glucosidase inhibitors, and/or insulin sensitizers (e.g., one or more glitazones—such as rosiglitazone and/or pioglitazone)).

IRBPs typically are used in and provided in an at least substantially pure form. A substantially pure molecule is a molecule that is the predominant species in the composition wherein it is found with respect to the class of molecules to which it belongs (e.g., a substantially pure protein is the predominant protein species in the composition wherein it is found). A substantially pure species makes up at least about 50% of the type of molecule in the composition and typically will make up at least about 70%, at least about 80%, at least about 85%, at least about 90%, at least about 95%, or greater percentage of the molecular species in the composition by weight. Commonly, a composition comprising an IRBP will exhibit at least about 98%, 98%, or 99% homogeneity for the IRBP in the context of all present peptide species in the composition or at least with respect to substantially active peptide species in the context of proposed use. For example, a peptide stabilizer/buffer such as an albumin may be intentionally included in a final pharmaceutical formulation, without impeding the activity of the IRBPs, and, accordingly, may be excluded from such purity calculations. The presence of impurities that do not interfere with the fundamental activity also may be acceptable in the context of a substantially pure composition. Purity can be measured by methods appropriate for the given compound (e.g., chromatographic methods; agarose and/or polyacrylamide gel electrophoresis; HPLC analysis; etc.).

An isolated molecule refers to a molecule that is not associated with significant levels (e.g., more than about 1%, more than about 2%, more than about 3%, or more than about 5%) of any extraneous and undesirable biological molecules, such as non-IRBP biological molecules contained within a cell, cell culture, chemical media, or animal in which the IRBP was produced. An isolated molecule also refers to any molecule that has passed through such a stage of purity due to human intervention (whether automatic, manual, or both) for a significant amount of time (e.g., at least about 10 minutes, at least about 20 minutes, at least about one hour, or longer). In many of the various compositions provided by the invention, such as in a composition comprising one or more pharmaceutically acceptable carriers, a IRBP can be present in relatively small amounts in terms of numbers of total molecular species in the composition (e.g., in the case of a composition comprising a large amount of a pharmaceutically acceptable carrier, stabilizer, and/or preservative). In some cases additional peptides, such as BSA, can be included in such a composition with a previously purified IRBP. However, provided that such additional constituents of the composition are acceptable for the intended application of the IRBP, such a composition can still be described as comprising an isolated IRBP. In other words, the term “isolated” is not meant to exclude artificial or synthetic mixtures with other compounds or materials, such as may form part of a pharmaceutically acceptable preparation.

In one aspect, the invention provides IRBPs that are substantially free of other IR-binding molecules.

In another aspect, the invention provides a composition comprising a number of IRBPs with different specificities and characteristics (e.g., the invention provides in one aspect a “cocktail” of IRBPs having different affinity, stability, specificity and/or selectivity characteristics).

IRBP compositions for pharmaceutical use typically contain at least a physiologically effective amount and commonly desirably contain a therapeutically effective amount of an IRBP, combination of IRBPs, or one or more IRBP(s) and additional active/therapeutic agents.

A “therapeutically effective amount” refers to an amount of a biologically active compound or composition that, when delivered in appropriate dosages and for appropriate periods of time to a host that typically is responsive for the compound or composition, is sufficient to achieve a desired therapeutic result in a host and/or typically able to achieve such a therapeutic result in substantially similar hosts (e.g., patients having similar characteristics as a patient to be treated). A therapeutically effective amount of an IRBP may vary according to factors such as the disease state, age, sex, and weight of the individual, and the ability of the IRBP to elicit a desired response in the individual. A therapeutically effective amount is also one in which any toxic or detrimental effects of the agent are outweighed by the therapeutically beneficial effects. Exemplary therapeutic effects include, e.g., (a) a reduction in the severity of a disease, disorder, or related condition in a particular subject or a population of substantial similar subject; (b) a reduction in one or more symptoms or physiological conditions associated with a disease, disorder, or condition; and/or (c) a prophylactic effect. A reduction of the severity of a disease can include, for example, (a) a measurable reduction in the spread of a disorder; (b) an increase in the chance of a positive outcome in a subject (e.g., an increase of at least about 5%, 10%, 15%, 20%, 25%, or more); (c) an increased chance of survival or lifespan; and/or (d) a measurable reduction in one or more biomarkers associated with the presence of the disease state (e.g., a reduction in the amount and/or severity of diabetic symptoms; etc.). A therapeutically effective amount can be measured in the context of an individual subject or, more commonly, in the context of a population of substantial similar subjects (e.g., a number of human patients with a similar disorder enrolled in a clinical trial involving a IRBP composition or a number of non-human mammals having a similar set of characteristics being used to test a IRBP in the context of preclinical experiments).

IRBPs also can be delivered to a host in a prophylactically effective amount as part of a disease/disorder prevention program or for otherwise increasing general health. A “prophylactically effective amount” refers to an amount of an active compound or composition that is effective, at dosages and for periods of time necessary, in a host typically responsive to such compound or composition, to achieve a desired prophylactic result in a host or typically able to achieve such results in substantially similar hosts. Exemplary prophylactic effects include a reduction in the likelihood of developing a disorder, a reduction in the intensity or spread of a disorder, an increase in the likelihood of survival during an imminent disorder, a delay in the onset of a disease condition, a decrease in the spread of an imminent condition as compared to in similar patients not receiving the prophylactic regimen, etc. Typically, because a prophylactic dose is used in subjects prior to or at an earlier stage of disease, the prophylactically effective amount will be less than the therapeutically effective amount for a particular IRBP. A prophylactic effect also can include, e.g., a prevention of the onset, a delay in the time to onset, a reduction in the consequent severity of the disease as compared to a substantially similar subject not receiving IRBP composition, etc.

In another aspect, IRBPs can be delivered to a host or cells in a physiologically effective amount. A physiologically effective amount is an amount of an active agent that upon administration to a host that is normally responsive to such an agent results in the induction, promotion, and/or enhancement of at least one physiological effect associated with modulation of IR activity (e.g., modulation of IR phosphorylation, reduction in blood glucose levels, and/or IR-associated signaling).

“Treatment” refers to the delivery of an effective amount of a therapeutically active compound of the invention with the purpose of preventing any symptoms or disease state to develop or with the purpose of easing, ameliorating, or eradicating (curing) such symptoms or disease states already developed. The term “treatment” is thus meant to include prophylactic treatment. However, it will be understood that therapeutic regimens and prophylactic regimens of the invention also can be considered separate and independent aspects of this invention.

1. Pharmaceutically Acceptable Carriers

An IRBP can be combined with one or more carriers (diluents, excipients, and the like) appropriate for one or more intended routes of administration to provide compositions that are pharmaceutically acceptable in the context of preparing a pharmaceutically acceptable composition comprising one or more IRBPs.

IRBPs may be, for example, admixed with lactose, sucrose, powders (e.g., starch powder), cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and optionally further tabletted or encapsulated for conventional administration. Alternatively, an IRBP may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other carriers, adjuvants, and modes of administration are well known in the pharmaceutical arts. A carrier or diluent may include time delay material, such as glyceryl monostearate or glyceryl distearate alone or with a wax, or other functionally similar materials.

Pharmaceutically acceptable carriers generally also include any and all suitable solvents, dispersion media, coatings, antibacterial and antifungal agents, isotonic and absorption delaying agents, and the like that are physiologically compatible with a IRBP. Examples of pharmaceutically acceptable carriers include water, saline, phosphate buffered saline (PBS), dextrose, glycerol, ethanol, and the like, as well as combinations of any thereof. In many cases, it can be desirable to include isotonic agents, for example, sugars, polyalcohols such as mannitol, sorbitol, or sodium chloride in such a composition. Pharmaceutically acceptable substances such as wetting agents or minor amounts of auxiliary substances such as wetting agents or emulsifying agents, preservatives or buffers, which desirably can enhance the shelf life or effectiveness of the IRBP, related composition, or combination. Suitability for carriers and other components of pharmaceutical compositions can be determined based on the lack of significant negative impact on the desired biological properties of the IRBP, related composition, or combination (e.g., less than an about 20%, 15%, 10%, 5%, or 1% reduction in IR binding and/or activation; or ability to reduce blood glucose in a target host).

IRBP compositions, related compositions, and combinations according to the invention may be presented, prepared, and/or administered in a variety of suitable forms. Such forms include, for example, liquid, semi-solid and solid dosage forms, such as liquid solutions (e.g., injectable and infusible solutions), dispersions or suspensions, emulsions, microemulsions, tablets, pills, powders, liposomes, dendrimers and other nanoparticles (see, e.g., Baek et al., Methods Enzymol. 2003; 362:240-9; Nigavekar et al., Pharm Res. 2004 March; 21(3):476-83), microparticles, and suppositories. The optimal form for any IRBP-associated composition depends on the intended mode of administration, the nature of the composition or combination, and therapeutic application or other intended use. Formulations also can include, for example, powders, pastes, ointments, jellies, waxes, oils, lipids, lipid (cationic or anionic) containing vesicles, DNA conjugates, anhydrous absorption pastes, oil-in-water and water-in-oil emulsions, emulsions, carbowax (polyethylene glycols of various molecular weights), semi-solid gels, and semi-solid mixtures containing carbowax. Any of the foregoing mixtures may be appropriate in treatments and therapies in accordance with the present invention, provided that the binding of the IRBP to cognate IRs is not significantly inhibited by the formulation and the formulation is physiologically compatible and tolerable with the route of administration. See also, e.g., Powell et al. “Compendium of excipients for parenteral formulations” PDA J Pharm Sci Technol. 52:238-311 (1998) and the citations therein for additional information related to excipients and carriers well known to pharmaceutical chemists.

In a particular aspect, IRBPs are administered in liposomes. In another aspect, IRBPs are administered in liposomes with one or more secondary agents, such as one or more anti-diabetes drugs.

IRBP compositions also include compositions comprising any suitable combination of an IRBP peptide and a suitable salt therefor. Any suitable salt, such as an alkaline earth metal salt in any suitable form (e.g., a buffer salt), can be used in the stabilization of IRBPs (preferably the amount of salt is such that oxidation and/or precipitation of the IRBP is avoided). Suitable salts typically include sodium chloride, sodium succinate, sodium sulfate, potassium chloride, magnesium chloride, magnesium sulfate, and calcium chloride. Compositions comprising a base and one or more IRBPs also are provided.

A typical mode for delivery of IRBP compositions is by parenteral administration (e.g., intravenous, subcutaneous, intraperitoneal, and/or intramuscular administration). In one aspect, an IRBP is administered to a human patient by intravenous infusion or injection. In another aspect, an IRBP is administered by intramuscular or subcutaneous injection. As indicated above, intratumor administration also may be useful in certain therapeutic regimens.

Thus, IRBPs may be formulated in, for example, solid formulations (including, e.g., granules, powders, projectile particles, or suppositories), semisolid forms (gels, creams, etc.), or in liquid forms (e.g., solutions, suspension, or emulsions). IRBPs may be applied in a variety of solutions. Suitable solutions for use in accordance with the invention typically are sterile, dissolve sufficient amounts of the IRBP and other components of the composition, stable under conditions for manufacture and storage, and not harmful to the subject for the proposed application. An IRBP may be subjected to conventional pharmaceutical operations such as sterilization and/or may contain conventional adjuvants, such as preservatives, stabilizers, wetting agents, emulsifiers, buffers etc. A composition also can be formulated as a solution, microemulsion, dispersion, powder, macroemulsion, liposome, or other ordered structure suitable to high drug concentration. Desirable fluidity properties of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin. These and other components of a pharmaceutically acceptable composition of the invention can impart advantageous properties such as improved transfer, delivery, tolerance, and the like.

A composition for pharmaceutical use can include various diluents, fillers, salts, buffers, detergents (e.g., a nonionic detergent, such as Tween-80), stabilizers (e.g., sugars or protein-free amino acids), preservatives, tissue fixatives, solubilizers, and/or other materials suitable for inclusion in a composition for pharmaceutical use. Examples of suitable components also are described in, e.g., Berge et al., J. Pharm. Sci., 6661), 1-19 (1977); Wang and Hanson, J. Parenteral. Sci. Tech: 42, S4-S6 (1988); U.S. Pat. Nos. 6,165,779 and 6,225,289; and other documents cited herein. Such a pharmaceutical composition also can include preservatives, antioxidants, or other additives known to those of skill in the art. Additional pharmaceutically acceptable carriers are known in the art and described in, e.g., Urquhart et al., Lancet, 16, 367 (1980), Lieberman et al., Pharmaceutical Dosage Forms—Disperse Systems (2nd ed., vol. 3,1998); Ansel et al., Pharmaceutical Dosage Forms & Drug Delivery Systems (7th ed. 2000); Martindale, The Extra Pharmacopeia (31st edition), Remington's Pharmaceutical Sciences (16th-20th editions); The Pharmacological Basis Of Therapeutics, Goodman and Gilman, Eds. (9th ed.-1996); Wilson and Gisvolds' TEXTBOOK OF ORGANIC MEDICINAL AND PHARMACEUTICAL CHEMISTRY, Delgado and Remers, Eds. (10th ed.—1998), and U.S. Pat. Nos. 5,708,025 and 5,994,106. Principles of formulating pharmaceutically acceptable compositions also are described in, e.g., Platt, Clin. Lab Med., 7:289-99 (1987), Aulton, Pharmaceutics: The Science Of Dosage Form Design, Churchill Livingstone (New York) (1988), EXTEMPORANEOUS ORAL LIQUID DOSAGE PREPARATIONS, CSHP (1998), and “Drug Dosage,” J. Kans. Med. Soc., 70 (I), 30-32 (1969). Additional pharmaceutically acceptable carriers particularly suitable for administration of IRBP compositions and related compositions (e.g., compositions comprising IRBP-encoding nucleic acids or IRBP-encoding nucleic acid comprising vectors) are described in, for example, International Patent Application WO 98/32859.

IRBP compositions can be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid, and combinations of any thereof, so as to provide such a composition. Methods for the preparation of such compositions are known. See, e.g., Sustained and Controlled Release Drug Delivery Systems, J. R. Robinson, ed., Marcel Dekker, Inc., New York, 1978.

In another aspect, compositions of the invention orally administered, for example, with an inert diluent or an assimilable edible carrier (specific oral administration formulations and methods are also separately described elsewhere herein). The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

The preparation of pharmaceutical compositions that contain peptides as active ingredients is well understood in the art. Typically, such compositions are prepared as injectables, either as liquid solutions or suspensions, however, solid forms suitable for solution in, or suspension in, liquid prior to injection can also be prepared. The preparation can also be emulsified. The active therapeutic ingredient is often mixed with excipients that are pharmaceutically (i.e., physiologically) acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH-buffering agents, which enhance the effectiveness of the active ingredient.

An IRBP can be formulated into a pharmaceutical composition as neutralized physiologically acceptable salt forms. Suitable salts include the acid addition salts (i.e., formed with the free amino groups of the peptide molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In the case of combination compositions (discussed further herein), IRBPs can be coformulated with and/or coadministered with one or more additional therapeutic agents (e.g., an anti-diabetic agent such as an insulin, an insulin analogue, metformin or other anti-diabetic biguanide, a glucagon receptor antagonist, sulfonylurea, a thiazolidinedione, an alpha-glucosidase inhibitor, a meglitinide, a glucagon-like peptide-1 (GLP-1), a GLP-1 analog, etc.). Such combination therapies may require lower dosages of the IRBP and/or the co-administered agents, so as to avoid possible toxicities or complications associated with the various monotherapies.

D. Therapeutic Applications

As indicated above, IRBPs can be administered individually or in combination with other pharmacologically active agents. It will be understood that such combination therapy encompasses different therapeutic regimens, including, without limitation, administration of multiple agents together in a single dosage form or in distinct, individual dosage forms. If the agents are present in different dosage forms, administration may be simultaneous or near-simultaneous or may follow any predetermined regimen that encompasses administration of the different agents.

For example, when used to treat diabetes or other diseases or syndromes associated with a decreased response or production of insulin, hyperlipidemia, obesity, appetite-related syndromes, and the like, the peptides of the invention may be advantageously administered in a combination treatment regimen with one or more agents, including, without limitation, insulin, insulin analogues, insulin derivatives, glucagon-like peptide-1 or -2 (GLP-1, GLP-2), derivatives or analogues of GLP-1 or GLP-2 (such as are disclosed, e.g., in WO 00/55119). It will be understood that an “analogue” of insulin, GLP-1, or GLP-2 as used herein refers to a peptide containing one or more amino acid substitutions relative to the native sequence of insulin, GLP-1, or GLP-2, as applicable; and “derivative” of insulin, GLP-1, or GLP-2 as used herein refers to a native or analogue insulin, GLP-1, or GLP-2 peptide that has undergone one or more additional chemical modifications of the amino acid sequence, in particular relative to the natural sequence. Insulin derivatives and analogues are disclosed, e.g., in U.S. Pat. Nos. 5,656,722, 5,750,497, 6,251,856, and 6,268,335. In some embodiments, the combination agent is one of Lys^{B29}(ε-myristoyl)des(B30) human insulin, Lys^{B29}(ε-tetradecanoyl)des(B30) human insulin and B²⁹—N^{ε}—(N-lithocolyl-γ-glutamyl)-des(B30) human insulin. Also suitable for combination therapy are non-peptide antihyperglycemic agents, antihyperlipidemic agents, and the like such as those well-known in the art.

In one embodiment, the invention encompasses methods of treating diabetes or related syndromes or associated conditions (e.g., reducing the rate of glucose level-related and/or diabetes-related stroke, heart disease, kidney disease, blindness, and/or loss of sensation in the limbs) comprising delivering an effective amount of at least one IRBP (by gene expression, by delivery of a homogenous peptide, or typically by administration of a pharmaceutically acceptable composition comprising one or more IRBPs and one or more pharmaceutically acceptable carriers as described above. In another aspect, the invention provides the use of an IRBP or IRBP composition (such as a combination composition) in the manufacture of a medicament used in the treatment of type 1 or type 2 diabetes.

IRBPs can generally be used in the treatment of both Type 1 and Type 2, i.e., insulin dependent diabetes mellitus (IDDM) and non-insulin dependent diabetes mellitus (NIDDM).

In an exemplary combination therapy aspect, the invention provides a method of treating diabetes (e.g., reducing one or more symptoms associated therewith in a host and/or providing to a host an amount of a composition that has been demonstrated to be therapeutically effective in at least a substantial proportion of a population of similar hosts) comprising delivering a first amount of a IRBP and a second amount of a long-acting insulin analogue, such as, e.g., Lys^{B29}(ε-myristoyl)des(B30) human insulin, Lys^{B29}(ε-tetradecanoyl)des(B30) human insulin or B²⁹—N^{ε}—(N-lithocolyl-γ-glutamyl)-des(B30) human insulin, wherein the first and second amounts together are effective for treating the syndrome. As used herein, a long-acting insulin analogue is one that exhibits a protracted profile of action relative to native human insulin, as disclosed, e.g., in U.S. Pat. No. 6,451,970. In another aspect, the invention provides the use of a combination composition comprising a therapeutically effective combination of at least one IRBP and at least one insulin or insulin analog in the manufacture of a medicament used in the treatment of disease, such as in the treatment of type 1 or type 2 diabetes. Similar compositions comprising combinations of one or more IRBPs and one or more long and/or short-acting insulin analogs also can be suitable for therapeutic methods, such as the treatment of diabetes.

In one aspect, the invention provides a method provides a method of treating symptoms and/or underlying conditions associated with Type 2 diabetes in a patient in need thereof (due to diagnosis of the disease and/or substantial risk of development thereof) comprising delivering to the patient a therapeutically and/or prophylactically effective amount of an IRBP or IRBP composition of the invention so as to treat such symptoms and/or conditions. In a particular aspect, the invention provides a method of treating a patient having Type 2 diabetes and high insulin blood levels (hyperinsulinemia). In one such aspect, the patient is obese. In another aspect, the patient also or alternatively comprises an insulin resistant genotype/mutation.

In another aspect, the invention provides a method of reducing blood pressure in a patient having insulin/IR-associated high blood pressure comprising administering or otherwise delivering a therapeutically effective amount of an IRBP or IRBP composition of the invention so as to reduce blood pressure in the patient.

In another aspect, the invention provides a method of treating the symptoms and/or underlying conditions of Syndrome X or an aspect thereof in a patient (e.g., hyperlipdemia, hypertension, and/or obesity) comprising administering or otherwise delivering a therapeutically and/or prophylactically effective amount of an IRBP of the invention to the patient so as to treat Syndrome X or a Syndrome X condition.

In still another aspect, the invention provides a method of treating a non-diabetes IR-mediated condition, disorder, or disease in a patient, such as an IR-associated neurodegenerative disease; an IR-associated non-diabetes autoimmune disease; etc., comprising administering a therapeutically or prophylactically effective amount of an IRBP or IRBP composition of the invention to the patient to treat such conditions/symptoms.

In another aspect, the invention provides a method of preventing weight gain in a patient in need thereof comprising administering a therapeutically or prophylactically effective amount of an IRBP or IRBP composition of the invention to the patient so as to prevent IR-associated weight gain.

In a related aspect, the invention provides a method of treating obesity comprising administering a therapeutically or prophylactically effective amount of an IRBP or IRBP composition of the invention to the patient to treat obesity (by stabilizing and/or reducing the weight of the patient) or a condition related thereto.

In another aspect, the invention provides a method of treating a patient suffering from a disease condition associated with or caused by hyperinsulinaemia, hypoglycaemia, hypokalaemia, and/or hypophosphataemia comprising administering (or otherwise delivering—as is the case throughout) a therapeutically or prophylactically effective amount of an IRBP or IRBP composition of the invention to the patient to treat such conditions/symptoms.

In another aspect, the invention provides the use of an IRBP or IRBP composition (such as a combination composition) in the manufacture of a medicament used in the treatment of any of the foregoing conditions.

In one general aspect, the invention provides a method of modulating glucose levels in an individual comprising administering a physiologically effective amount of an IRBP or IRBP composition of the invention so as to detectably modulate glucose levels in the patient. In another aspect, the invention provides the use of an IRBP or IRBP composition (such as a combination composition) in the manufacture of a medicament used in the reducing of blood glucose levels.

In another general aspect, the invention provides a method of mediating IR activity comprising administering a physiologically effective amount of an IRBP or IRBP composition of the invention such that responsive IR on IR-presenting cells is bound in an amount and under conditions sufficient to induce, promote, enhance, and/or otherwise modulate an IR-mediated activity or response. For example, IRBPs can be delivered to a host to bind to Site 1 or Site 2, so as to direct insulin or insulin analog molecules to the other site so as to modify the profile of an insulin or insulin analog treatment.

In yet a further facet, the invention provides a method of modulating nitric oxide production levels in a patient, such as in the endothelial cells of a patient; mediating RAS, RAF, MEK, and/or mitogen-activated protein (MAP) kinase pathways; modulating vascular tissue growth and/or smooth muscle cell, monocyte, macrophage, and/or endothelial cell growth and/or migration; stimulate production of plasminogen activator inhibitor type 1 (PAI-1); modulate endothelin production; modulate IR-associated proatherosclerotic pathway biological events; modulate IR-associated inflammation; treat and/or reduce the risk of arterial injury; treat and/or prevent atherosclerosis; and/or reduce IR-associated inflammation molecules, such as LDL cholesterol in blood vessel walls of a patient by delivering or otherwise administering a therapeutically or prophylactically effective amount of an IRBP or IRBP composition of the invention to the patient to induce, promote, and/or enhance such physiological responses.

1. Delivery and Administration Methods

In general, IRBPs can be delivered by any suitable manner, such as by expression from a nucleic acid that codes for production of the IRBP in target host cells (e.g., by expression from a IRBP-encoding nucleic acid under the control of an inducible promoter and comprised in a suitable gene transfer vector, such as a targeted and replication-deficient gene transfer vector). Typically, IRBPs are delivered by direct administration of the IRBP or IRBP composition to a recipient host. Thus, IRBPs and IRBP compositions may be administered as pharmaceutical compositions comprising standard carriers known in the art for delivering proteins and peptides and/or delivered by gene therapy. In general and where appropriate the terms administration and delivery should be construed as providing support for one another herein (e.g., it should generally be recognized that IRBP-encoding nucleic acids can be used to deliver naked IRBPs to target host tissues as an alternative to administration of IRBP proteins), although it also should be recognized that each such method is a unique aspect of the invention with respect to any particular molecule and that some molecules (e.g., conjugated IRBPs comprising degradation-resistant organic moieties) are amenable to only certain forms of delivery/administration. Methods for the administration of proteins, nucleic acids, and related compositions (e.g., vectors and host cells), are well known and, accordingly, only briefly described here.

IRBP compositions, related compositions, and combination compositions can be administered via any suitable route, such as an oral, mucosal, buccal, intranasal, inhalable, intravenous, subcutaneous, intramuscular, parenteral, or topical route. Such proteins may also be administered continuously via a minipump or other suitable device.

An IRBP or other IRBP generally will be administered for as long as the disease condition is present, provided that the protein causes the condition to stop worsening or to improve. The IRBP will generally be administered as part of a pharmaceutically acceptable composition, e.g., as described in detail elsewhere herein.

An IRBP may also be administered or otherwise delivered prophylactically to prevent a disease, disorder, or condition for which such treatment may be effective. For example, IRBPs can be administered or otherwise delivered to a patient in remission from a serious diabetic condition (e.g., a significant risk of the onset of diabetes-associated blindness, amputation, or other condition, etc.) in order to reduce the risk of the risk of recurrence diabetes-associated condition.

In general, a IRBP or other IRBP (or related composition such as a vector comprising a IRBP-encoding nucleic acid) may be administered by any suitable route, but typically is administered parenterally in dosage unit formulations containing conventional pharmaceutically acceptable carriers, adjuvants, and the like (stabilizers, disintegrating agents, anti-oxidants, etc.). The term “parenteral” as used herein includes, subcutaneous, intravenous, intraarterial, intramuscular, intrasternal, intratendinous, intraspinal, intracranial, intrathoracic, infusion techniques and intraperitoneal delivery. Thus, in one aspect, an IRBP composition is administered intravenously or subcutaneously, in practicing therapeutic methods of the invention. Routes of injection also include injection into the muscle (intramuscular IM); injection under the skin (subcutaneous (s.c.)); injection into a vein (intravenous (IV)); injection into the abdominal cavity (intraperitoneal (IP)); and other delivery into/through the skin (intradermal delivery, usually by multiple injections, which may include biolistic injections).

In one aspect the invention provides a method of modulating IR activity in a host comprising administering a pharmaceutical composition that includes, in admixture, a pharmaceutically (i.e., physiologically) acceptable carrier, excipient, or diluent, and one or more IR agonist IRBPs as an active agent component (which may be further combined with secondary active agents as described elsewhere).

The pharmaceutical compositions of the invention can be administered systemically by oral or parenteral routes. Non-limiting parenteral routes of administration include subcutaneous, intramuscular, intraperitoneal, intravenous, transdermal, inhalation, intranasal, intra-arterial, intrathecal, enteral, sublingual, or rectal. Due to the labile nature of typical amino acid sequences parenteral administration may be advantageous. Advantageous modes of administration include, e.g., aerosols for nasal or bronchial absorption; suspensions for intravenous, intramuscular, intrasternal or subcutaneous, injection; and compounds for oral administration.

Intravenous administration, for example, can be performed by injection of a unit dose. The term “unit dose” when used in reference to a pharmaceutical composition of the present invention refers to physically discrete units suitable as unitary dosage for humans, each unit containing a predetermined quantity of active material calculated to produce the desired therapeutic effect in association with the required diluent; i.e., liquid used to dilute a concentrated or pure substance (either liquid or solid), making that substance the correct (diluted) concentration for use. For injectable administration, the composition is in sterile solution or suspension or may be emulsified in pharmaceutically- and physiologically-acceptable aqueous or oleaginous vehicles, which may contain preservatives, stabilizers, and material for rendering the solution or suspension isotonic with body fluids (i.e., blood) of the recipient.

Excipients suitable for use are water, phosphate buffered saline, aqueous sodium chloride solution, dextrose, glycerol, dilute ethanol, and the like, and mixtures thereof. Illustrative stabilizers are polyethylene glycol, proteins, saccharides, amino acids, inorganic acids, and organic acids, which may be used either on their own or as admixtures. The amounts or quantities, as well as routes of administration, used are determined on an individual basis, and correspond to the amounts used in similar types of applications or indications known to those of skill in the art.

Pharmaceutical compositions can typically be administered in a manner compatible with the dosage formulation, and in a therapeutically effective amount. The quantity to be administered depends on the subject to be treated, capacity of the subject's immune system to utilize the active ingredient, and degree and type of modulation of IR desired. Precise amounts of active ingredient required to be administered depend on the judgment of the practitioner and are specific for each individual. However, suitable dosages may range from about 10 to 200 nmol active peptide per kilogram body weight of individual per day and depend on the route of administration. Suitable regimes for initial administration and booster shots are also variable, but are typified by an initial administration followed by repeated doses at one or more hour intervals by a subsequent injection or other administration. Alternatively, continuous intravenous infusions sufficient to maintain picomolar concentrations (e.g., approximately 1 pM to approximately 10 nM) in the blood are contemplated. An exemplary formulation comprises an IR agonist IRBP in a mixture with sodium busulfite USP (3.2 mg/ml); disodium edetate USP (0.1 mg/ml); and water for injection q.s.a.d. (1 ml).

In another particular aspect, an IRBP or an IRBP composition is delivered by an injectable pump in a liquid or other suitable formulation for use with such devices. IRPBs also can be administered by pens, such as are currently used to deliver insulin products. The use of transdermal patches (e.g., a drug in matrix patch) also can be used to deliver IRBPs (e.g., by passive delivery or via iontophoretic delivery).

Further guidance in preparing pharmaceutical formulations can be found in, e.g., Gilman et al. (eds), 1990, Goodman and Gilman's: The Pharmacological Basis of Therapeutics, 8th ed., Pergamon Press; and Remington's Pharmaceutical Sciences, 17th ed., 1990, Mack Publishing Co., Easton, Pa.; Avis et al. (eds), 1993, Pharmaceutical Dosage Forms: Parenteral Medications, Dekker, New York; Lieberman et al. (eds), 1990, Pharmaceutical Dosage Forms: Disperse Systems, Dekker, New York.

a. Exemplary Dosages and Administration Strategies

As described above, compositions of the invention may include a “therapeutically effective amount” or a “prophylactically effective amount” of a IRBP (or first and second amounts in the case of a combination composition comprising a IRBP and a second component; first, second, and third amounts in the case of a combination composition comprising two IRBPs and a secondary agent or a IRBP and two secondary agents; etc.). To better illustrate particular aspects, a detailed discussion of dosage principles is further provided here.

In practicing the invention, the amount or dosage range of the IRBP employed typically is one that effectively induces, promotes, or enhances a physiological response associated with IRBP binding of a cognate IR. In one aspect, the dosage range is selected such that the IRBP employed induces, promotes, or enhances a medially significant effect in a patient suffering from or being at substantial risk of developing a condition associated that is at least in part modulated by IR activity, such as, e.g., a form of diabetes, which effect is associated with the activation, signaling, and/or biological modification (e.g., phosphorylation) of the cognate IR.

In still another aspect, a daily dosage of active ingredient (e.g., IRBP) of about 0.01 to 100 milligrams per kilogram of body weight is provided to a patient. Ordinarily, about 1 to about 5 or about 1 to about 10 milligrams per kilogram per day given in divided doses of about 1 to about 6 times a day or in sustained release form may be effective to obtain desired results.

As a non-limiting example, treatment of IR-associated pathologies in humans or animals can be provided by administration of a daily dosage of IRBP(s) in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20, or any combination thereof, using single or divided doses of every about 24, 12, 8, 6, 4, or 2 hours, or any combination thereof.

In one aspect, the inventive methods comprise administering or otherwise delivering two different IRBPs over a period of one month, the beginning of the therapy involving the second IRBP starting about 1-3 weeks (e.g., about 10 days) after the first delivery of the first IRBP or at any time when a significant immune response to the first IRBP develops in the host, such that the continued use of the first IRBP has become detrimental to the patient.

b. Oral Delivery Formulations

A particularly advantageous aspect of the invention is embodied in a pharmaceutically acceptable composition comprising a therapeutically and/or prophylactically effective amount of one or more digestive enzyme stabilized IRBPs (comprising one or more unusual degradation-resistant amino acid residues and/or degradation resistant moieties as described above) formulated for oral administration and the use of such a composition in the modulation of IR activity (e.g., in the context of treating diabetes or a related condition, such as IR-modulated metabolic disorder). The relatively small size of typical IRBPs and simple structure (e.g., a single chain of about 30 amino acid residues in the case of many IRBP dimers and a single chain of about 5-20 residues in the case of monomers) in and of itself is believed to aid in the oral delivery of such proteins as compared to human insulin (which is a two chain peptide of 51 residues and often delivered in higher-ordered multimeric forms). The addition of N- and/or C-terminal blocking modifications (particularly, e.g., acetylation and amidation, respectively) are believed to increase the ability of such molecules to be delivered orally as compared to insulin. The inclusion of degradation-resistant unusual amino acid residues and/or organic moieties also or alternatively is believed to significantly increase the ability of such peptides to be delivered effectively by an oral route as compared to presently known forms of insulins and insulin analogs. IRBPs comprising a combination of such features are believed to be particularly advantageous for oral delivery suitable anti-diabetic agents (e.g., IRBPs comprising at least one, typically at least two degradation-resistant residues and/or moieties—such as one or more D-arginine residues—and having at least N- or C-terminal blockage, typically both by respective acetylation and amidation are expected to be particularly suitable for oral administration).

In one aspect, the invention provides IRBP oral formulations that may exhibit at least about 1%, at least about 5%, at least about 10%, at least about 15%, at least about 20% or more relative bioavailability upon oral administration as compared to parenteral injection. The inherent oral delivery capacity of IRBPs can be enhanced by formulating the IRBPs with oral delivery enhancing compositions using methods known in the art and that have been demonstrated to be effective in enhancing the oral delivery and availability of small peptides (e.g., peptides of the size of insulin and smaller, but over 5 amino acids in length). Such compositions are another important facet of the invention.

In general, oral formulations seek to inhibit or modulate proteolytic activity that degrades the peptide; enhance paracellular and/or transcellular transport of the peptide; improve peptide penetration through the mucus barrier (particularly in the case of fast-dissolving forms and aerosol-delivered or spray-delivered orally administered forms); and/or increase the half-life of the peptide in circulation (particularly for peptides that require a sustained presence for therapeutic efficacy). Devices can assist in such delivery. For example, IRBP compositions can be formulated for delivery by an aerosol spray device that allows delivery of the composition to the buccal mucosa and oropharynx region, wherein absorption of the formulation can occur. Examples of such devices, used for delivery of small peptides, such as insulin, are known in the art.

In one aspect, the invention provides an IRBP oral formulation composition wherein an IRBP is conjugated to a carrier molecule or encapsulated to improve stability of the IRBP as compared to the stability of the IRBP without the stabilizing conjugate or encapsulation materials(s). PEG conjugates and example of a typically stabilizing and delivery enhancing conjugate material. IRBPs can be, for example, conjugated to a PEG-based amphiphilic oligomer that increases GI absorption and/or reduces proteolytic degradation of the IRBP. In another exemplary aspect, calcium phosphate-PEG-insulin-casein (CAPIC) particles encasing IRBP compositions are used as an oral delivery form (microparticle and nanoparticle forms are further described elsewhere in this section).

In one aspect, an IRBP is conjugated to a delivery agent or carrier that facilitates passive transcellular transport. Desirably, such a carrier or agent is engineered so as to disassociate from the IRBP in circulation (e.g., upon reaching a certain level of exposure to low pH conditions).

In another aspect, an IRBP is formulated in an enteric-coated microcapsule or table containing one or more oral delivery facilitating excipients, such as sodium cholate and/or a trypsin inhibitor.

In yet a further aspect, an IRBP oral formulation is provided that comprises an effective amount of a detergent component that increases the solubility of the peptide, decreases interactions with intestinal mucus, and/or enhances paracellular transport.

In still another aspect, an IRBP is conjugated to one or more (typically several) low molecular weight (LMW) polymer conjugates that facilitate oral delivery by, e.g., adding resistance to enzymatic degradation with respect to related/similar naked IRBPs and/or allowing better gastrointestinal transport (e.g., improved diffusion through both water and fatty portions of cells and tissues that make up barriers to absorption along the gastrointestinal pathway and into the bloodstream).

Compositions, methods, and relevant principles for the construction of oral delivery-enhancing small peptide conjugates are provided in, e.g., U.S. Pat. Nos. 5,359,030; 5,438,040; 5,681,811; and 6,309,633.

In another aspect, the invention provides an oral delivery IRBP formulation that comprises an amino acid-based capsule system, which promotes intestinal lining passage and inhibits enzymatic degradation of the IRBP.

In still another aspect, the invention provides an IRBP oral delivery formulation that comprises a lipid or liposome encapsulation of an IRBP or IRBP composition, which promotes transmission through epithelial barrier(s) and/or protects the IRBP from enzymatic degradation. Desirably, such lipid formulations promote significant absorption of the pharmaceutical composition by the oral mucosa, thereby avoiding the “first pass” effect.

In another facet, the invention provides IRBP oral delivery formulations wherein an IRBP is contained within microparticles, which are administered directly or inserted in capsules, packets and the like for direct oral administration or administration by an oral delivery device (other oral delivery forms described herein generally can be delivered to a host by such methods as well). In a particular aspect, the invention provides an IRBP oral delivery formulation composed of alginate microspheres. Desirably, the alginate is a naturally occurring alginate that is classified as generally regarded as safe by the US FDA and optionally also induces or promotes a protective effect on the mucous membrane of the upper gastrointestinal tract. In another aspect, a coated crystal formulation is provided.

Extrusion spheronization is a technology that permits high concentrations of active substances to be included in high drug concentration pellets and that may be applicable to IRBP formulations.

In another aspect, an IRBP composition is presented as a dry syrup suspension of coated particles in a bottle or other container (e.g., a unit dose container) for liquid oral administration.

In general, formulations described herein can be sustained-release formulated, taste-masked or entero-coated compositions.

In another aspect, the invention provides a semi-solid matrix system in a relatively hard gelatin capsule for oral administration. Typically, such capsules further comprise a delivery promoting agent, such as a lipidic peptide delivery system. Soft pellet tablets comprising coated microparticles also are provided by the invention. Matrix tablets, which can be hydro-inert and/or lipo-inert, are another potentially suitable oral delivery form. Typically, such tables comprise coated microparticles of IRBP compositions and optionally peptidase inhibitors and/or penetration enhancers or other suitable excipients. Suitable penetration enhancers can include surfactants, fatty acids, bile salts, citrates, and chelators (e.g., EDTA), although other suitable penetration enhancers also can be included in these compositions or in other oral delivery forms described herein. For example, cyclodextrins may be used to enhance penetration of IRBP microparticles, conjugates, or other drug forms.

In an additional facet, the invention provides an IRBP composition comprising a bioadhesive polymer or other bioadhesive material, which typically facilitates association with the GI tract. Examples of such polymers include polycarbophil and chitosan.

As already mentioned, carrier systems, such as nanoparticles, microspheres, liposomes, and the like (e.g., small unilamellar vesicles (SUVs); albumin-containing nanoparticles; methylmethacrylate-containing nanoparticles; and albumin-containing microspheres (e.g., albumin/iron oxide magnetic and targetable microspheres or other targetable microparticle/nanoparticle formulations)) also or alternatively can be used to promote oral administration of an IRBP composition to a patient. Such formulations may be engineered to enhance absorption from the various regions of the GI tract and/or prevent degradation of the IRBP composition.

Emulsions and microemulsions also can be used as oral delivery formulations for IRBPs. For example, a water-in-oil emulsion comprising a hydrophobic phase comprising oleic acid, gadoleic acid, erucic acid, linoleic acid, linolenic acid, ricinoleic acid, arachidonic acid, glyceryl esters of such acids, oleyl alcohol, d-alpha-tocopherol polyethylene glycol succinate, combinations of any thereof, or similar molecules; a discontinuous aqueous hydrophilic phase; and at least one surfactant (e.g., poloxamer 124, a polyglycolized glyceride, sorbitan laurate, polyoxyethylene sorbitan monooleate, or similar surfactant can be included in such an emulsion and related pre-emulsion concentrate) for dispersing the hydrophilic phase (the hydrophobic phase typically forming about 5-10 wt. % of the emulsion) comprising the IRBP composition, which may include an alcohol, salt solution, etc.) in the hydrophobic phase (which typically is present in about 65-80 wt. % in the emulsion) as a water-in-oil emulsion may be useful in promoting oral delivery of IRBP compositions. Emulsions can be coated in enteric coating materials, which may be soluble in an acidic aqueous environment as mentioned with respect to other coating materials suitable for the oral delivery formulations of the invention.

In another aspect, the invention provides an oral delivery formulation comprising an IRBP associated with a thiolated polymer drug carrier matrix or polymer. An example of such a polymer is 2-Iminothiolane. In one aspect, such a polymer is covalently linked to chitosan to form a chitosan conjugate. Optionally, enzyme inhibitors (e.g., Bowman-Birk-Inhibitor and/or elastatinal) can be conjugated to the chitosan component of the conjugate. Also or alternatively, a permeation mediator can be included with the IRBP composition in tablets formed from such a chitosan. Immobilization of thiol groups on the polymer may enhance the mucoadhesive/cohesive properties of such formulations.

An encapsulation coat can include different combinations of pharmaceutical active ingredients, such as hydrophilic surfactants, lipophilic surfactants, and triglycerides. Sustained release oral delivery systems and/or enteric coatings for orally administered dosage forms are also contemplated such as those described in U.S. Pat. No. 4,704,295 issued Nov. 3, 1987, U.S. Pat. No. 4,556,552 issued Dec. 3, 1985, U.S. Pat. No. 4,309,404 issued Jan. 5, 1982 and U.S. Pat. No. 4,309,406 issued Jan. 5, 1982. Additional examples of solid carriers include bentonite, silica, and the like. Additional exemplary materials are described elsewhere herein.

In further aspect, IRBP oral delivery forms comprising hydrogels are provided. IRBPs can be incorporated and orally delivered in hydrogels of poly(methacrylic acid-g-ethylene glycol), for example. IRBPs also can be complexed with hydrogels. pH-responsive complexation hydrogels, such as hydrogels containing pendent glucose (P(MAA-co-MEG)) or grafted LMW (e.g., about 200 MW) PEG chains (P(MAA-g-EG)), may be advantageously used as IRBP oral delivery agents.

Nanospheres, microspheres, and other nanoparticles/microparticles can be formed from crosslinked networks of methacrylic acid and/or acrylic acid grafted with PEG(s) may be useful oral delivery formulations. Methods for entrapping drug product in such nanospheres at low pH (e.g., about 3), but as releasable agents at physiological pH (e.g., about 7) are known in the art. Additional exemplary microparticle formulations for naked and conjugated small peptide delivery and related methods and principles are set forth in, e.g., U.S. Pat. No. 6,191,105

An oral delivery formulation also can be presented as an inhalable composition selected from the group of aerosol, sprays and dry powders. Such pharmaceutical formulations of the present invention may be administered in the form of an aerosol spray using for example, a nebulizer such as those described in U.S. Pat. No. 4,624,251 issued Nov. 25, 1986; U.S. Pat. No. 3,703,173 issued Nov. 21, 1972; U.S. Pat. No. 3,561,444 issued Feb. 9, 1971 and U.S. Pat. No. 4,635,627 issued Jan. 13, 1971. Other systems of aerosol delivery, such as the pressurized metered does inhaler (MDI) and the dry powder inhaler as disclosed in Newman, S. P. in Aerosols and the Lung, Clarke, S. W. and Davia, D. eds. pp. 197-224, Butterworths, London, England, 1984, can be used when practicing the present invention.

In a further aspect, an IRBP composition is formulated as a mucoadhesive intestinal patch designed to deliver therapeutic doses of IRBP(s) into the systemic circulation of a patient. Such intestinal patches are thought to localize the associated IRBP near the mucosa and protect it from proteolytic degradation. Secure adhesion of such patches to the intestine has been demonstrated with insulin formulations and such patches have been shown to be effective for peptide drug delivery.

The oral delivery formulations described herein can be applied to the novel IRBPs specifically described herein, variants thereof (as described herein), IRBPs derived from the IRBPs described in the prior patent documents (as modified by one or more of the various aspects of this invention), or to even unmodified IRBPs described in the prior patent documents, provided that inclusion of such unmodified and previously characterized IRBPs in such compositions results in an increase in effectiveness in oral administration. The use of such compositions in the various methods of the invention is another facet of this invention.

c. Pulmonary Delivery Formulations

As already described herein, the invention also provides formulations for pulmonary delivery of IRBPs or IRBP compositions (e.g., compositions comprising combinations of IRBPs and/or IRBPs with secondary agents, such as secondary anti-diabetic agents, such as one or more insulins or insulin analogs). IRBPs can be directly administered to the lungs or administered in standard pharmaceutical formulations to the lungs (due to the above-described advantageous characteristics of such molecules for these forms of delivery), but, more typically, are administered in formulations engineered for pulmonary delivery, such as in particles that can be delivered as an aerosol for inhalation. IRBP compositions can be, for example, prepared as dry powders for administration by a dry powder (and stored in a suitable composition prior to delivery such as a blister pack) inhaler. The particle size of the particles in the dry formulation for such a system typically is less than about 5 μm in diameter and such particles typically are about 90% or more pure for drug composition. Such compositions can be prepared through known “glass stabilization techniques.” Alternatively, IRBP compositions can be formulated in aqueous compositions (also optionally stored in blister packs) and administered by an aqueous mist inhaler (e.g., a microprocessor controlled aqueous mist inhaler engineered to ensure proper dosing). Such devices can provide an increase in delivery of at least about 5-fold, such as about 10-fold, over a conventional nebulizer. Nebulizers, metered-dose inhalers (MDIs) and dry-powder inhalers (DPIs) can be useful in delivering such formulations. A number of such devices and similar devices have been developed for pulmonary delivery of peptide drugs. Formulations also can be engineered for long IR modulation activity upon delivery, such as, in the case of particle drugs, by using enhancing agents and/or by employing long action-promoting particle features (e.g., porous particles comprising large quantities, such as at least about 50% poly(lactic acid-co-glycolic acid); dry particles with a small aerodynamic size (e.g., about 1-3 μm), low density (e.g., about 0.1 gm/ml or less), and large geometric particle size (e.g., about 10-20 μm).

IRBP compositions may comprise IRBP derivatives, such as low molecular weight PEGylated IRBPs, that enhance the pulmonary delivery of such molecules. In another aspect, IRBP compositions are delivered to the lungs in the form of PEG particles, calcium phosphate (CAP) particles, or PEG-CAP particles (typically in suspension), which particles can be prepared using standard techniques (e.g., controlled precipitation techniques). Such particle compositions can be specifically delivered to the lungs by, e.g., intratracheal instillation and/or spray instillation. Such particles can also be optionally associated with one or more caseins (e.g., as a coating for PEG, CAP, or PEG-CAP particles—formed by adding the particles to a solution of caseins and permitting the caseins to aggregate around and/or complex with the particles). Similar compositions also can be useful for oral delivery forms or nasal delivery forms (e.g., oral spray formulations) and such molecules can be used in other forms (e.g., hydrogels).

A number of strategies, compositions, and devices for pulmonary and oral delivery of small peptides, such as insulin, have been developed and described in the art, that may also be applied to the IRBP and IRBP compositions of the invention (e.g., by modifying the IRBP similar to the insulin molecule described therein; formulating an IRBP composition similar to the insulin formulation described therein; administering the IRBP using methods and/or devices similar to those described therein; etc.). Examples of such methods, principles, delivery devices, and similar compositions that may be useful in preparation of such formulations are described in, e.g., Garcia-Contreras et al., AAPS PharmSci 2003; 5(2) Article 9 (2003); Steiner et al., Exp Clin Endocrinol Diabetes. 2002 January; 110(1):17-21; Pfutzner et al., Diabetes Technol Ther. 2002; 4(5):589-94; Gonda, I. et al.: Journal of Controlled Release 1998; 53:269-274; Schuster, J. et al.: Pharmaceutical Research 1997; 14(3):354-357; Farr, S. J. et al., Interpharm Press Inc., Buffalo Grove, Ill. 1996, pp. 175-184; Thippawong, J. et al., Diabetes Technology & Therapeutics 2002; 4(4): 499-504; Sangwan, S. et al., Journal of Aerosol Medicine. 2001; 14(2):185-195, Mudumba S. et al., Respiratory Drug Delivery VII. Dalby R. N. et al (eds) Serentec Press, Raleigh, N.C. 2000. pp. 329-332; Brinda, Curr Opin Investig Drugs. 2002 May; 3(5):758-62; Brunner, G. A. et al., Diabetologia 1991; 44:305-308; US Patent Applications 20040096401; 20040089290; 20030216542; 20030148925; 20030113273; 20020046750; 20010039260; 20030150446; and U.S. Pat. Nos. 6,635,617; 6,518,239; 6,349,719; 6,335,316; 6,098,615; 6,024,090; 5,672,581; 5,915,378; 5,970,240; and 5,813,358.

2. Combination Methods: Coadministration and Coapplication

As described above, the invention provides a number of combination compositions and combination methods (a combination method may include, e.g., a treatment plan that includes dietary modifications in a patient such as adopting a low glucose, low fat, and/or low glucose and low fat diet).

In combination administration/delivery methods, the dose and route of delivery of each of the IRBP and secondary agent(s) can be any suitable dosage and route for achieving the desired therapeutic, prophylactic, and/or physiological effects in the recipient host (e.g., lowering of blood glucose associated with IR activity modulation in a patient). In view of the combined effects of the IRBP and secondary agent in such methods and compositions, the dosage of the IRBP typically is lowered in such methods and compositions.

In general, combination administration methods of the invention can comprise any suitable administration scheme, including coadministration (as separate compositions or a single composition wherein the ingredients are mixed or separated) or stepwise administration of the various active agents.

The terms “coadministration,” “coadminister,” and the like herein refer to both to simultaneous administration (or concurrent administration) and serial but related administration, unless otherwise indicated. Coadministration of agents can be accomplished in any suitable manner and in any suitable time. In other words, coadministration can refer to administration of a IRBP before, simultaneously with, or after, the administration of a secondary agent, at any time(s) that result(s) in an enhancement in the therapeutic response over the administration of solely the secondary agent, IRBP, or both agents independently.

Treatment and or prophylactic regiments also can include coapplication of various methods in association with administration or deliver of an IRBP or IRBP composition (e.g., a combination composition as described herein), which may include, for example, application of a low glucose and/or low fat diet; application of an exercise regimen; application of an anti-diabetes gene therapy regimen; application of stem cell or other whole cell therapies (e.g., delivery of insulin-producing β cells—such as ex vivo engineered β cells); application of organ (e.g., pancreas) transplant; transplants of islets; provision of an integrated or connected insulin pump; etc.

When one or more agents are used in combination with an IRBP composition in a therapeutic regimen, there is no requirement for the combined results to be additive of the effects observed when each treatment is conducted separately. Although at least additive effects are generally desirable, any increased IR-mediated effect (e.g., anti-diabetes effect) above one of the single therapies would be of benefit. Also, there is no particular requirement for the combined treatment to exhibit synergistic effects, although this is certainly possible and typically advantageous.

To practice combined anti-diabetes therapy or therapy for other IR-associated condition, for example, one can simply administer to a mammal or other suitable animal an IRBP composition of this invention in combination with another anti-diabetes agent (e.g., GLP-1, a GLP-1 analog, a biguanide antidiabetic agent, a glucagon receptor antagonist, etc.) or application of a relevant therapeutic method (e.g., application of a diet therapy) in a manner effective to result in their combined anti-diabetes effect (e.g., reduction of one or more diabetes-associated symptoms and/or physiological conditions) in the treated animal. The agents or agent and method would therefore be provided or applied in amounts effective and for periods of time effective to result in a combined effect against the disease, disorder or condition. To achieve this goal, an IRBP composition and secondary agents/method may be administered or applied to the animal simultaneously, either in a single combined composition/method, or as two distinct compositions/methods using different administration routes (in the case of combination therapies).

In one exemplary aspect, an IRBP or IRBP composition is administered to a patient in association with application of an islet generation method, such as the administration/delivery of an islet-generating molecule, such as an islet-generating C-lectin protein, e.g., Islet Neogenesis Associated Protein (INGAP) or Reg (see, e.g., Kobayashi et al., J Biol Chem. 2000; 275:10723-10726 and Rafaeloff et al., J Clin Invest. 1997; 99:2100-2109).

Alternatively, the administration of an IRBP composition of this invention may precede, or follow, other associated anti-diabetes or other anti-disease therapy by, e.g., intervals ranging from minutes to weeks and months. One would ensure that the secondary anti-diabetes or anti-IR condition-associated agent and IRBP exert an advantageously combined effect on the condition, disorder, syndrome, etc.

As touched upon elsewhere herein, IRBPs can include one or more advantageous attributes that differ from human insulin including, for example, smaller size, longer in vivo half-life, greater resistance to degradation, greater bioavailability, and possibly greater insulin receptor affinity. IRBPs having one or more of such attributes also can be advantageously combined with insulins, insulin analogs, other insulin mimetics, and/or other anti-diabetes medications to provide a combination therapy or therapeutic composition that has a profile of action different from currently available therapeutic compositions.

In a particular aspect, the invention provides compositions that comprise one or more of S636, S642, S665, S726, S727, S733, and S873. In another particular aspect, the invention provides a method of modulating IR activity, or treating any of the diseases/disorders described herein, that comprises delivering one or more of these IRBPs to suitable host cells (in vitro or in vivo). In a further facet, the invention provides for the use of one or more of such IRBPs in the manufacture of a medicament to treat one or more the diseases or disorders described herein (e.g., type 1 and/or type 2 diabetes).

### Examples

The following examples are provided to further illustrate particular aspects of the invention but should not be understood as in any way limiting its scope.

### Example 1

This Example demonstrates a strategy for generating an enzymatically stable IRBP through enzymatic digestion analysis of a parent IRBP and subsequently introducing variations/derivations into a second, related IRBP to obtain a biologically similar IRBP with improved enzymatic stability as compared to the parent IRBP.

Previously described IRBP S597 was subjected to digestion with a series of relevant digestive enzymes (pepsin, elastase, chymotrypsin, trypsin, and carboxy-peptidase A) and the degradation products from such reactions were identified by standard mass spectrometry analysis thereby revealing the major cleavage sites in the IRBP by each enzyme.

A novel IRBP was derived from S597 by introducing the unusual amino acid residues aminoisobutyric acid and diphenylalanine into the Xaa₇ position of the N-terminal Formula 6-like IRBAAS sequence of S597 (in place of Ala) and into the Xaa₁ position of the C-terminal Formula 1 sequence (in place of Phe). The novel IRBP, obtained by these modifications, termed S873, was determined to exhibit IR affinity similar to insulin and exhibit greater than 100-fold stabilization towards all of the relevant enzymes as compared to S597.

A diagram illustrating the major points of enzymatic digestion in S597 and describing S873 is provided as FIG. 1 below.

This Example demonstrates how novel IRBPs of the invention possess improved stability with respect to enzymatic degradation, over previously characterized IRBPs lacking the same type and/or number of degradation-resistant residues and/or moieties, without losing relevant IR affinity.

This Example also demonstrates an additional aspect of the invention—the provision of a method for modifying an IRBP, such as an IRBP provided in the prior patent documents, to enhance enzyme degradation-resistance. Thus, application of such a method to other IRBPs disclosed in the prior patent documents embodies a further feature of this invention.

### Example 2

This Example demonstrates the preference of particular IRBPs of the invention for particular IR isoforms and IRs of particular species.

A number of IRBPs (S519; S557; S597; S636; S667; S733; S671; S660; S696; S574; S700; S626; and S726), some of which described in the prior patent documents, were applied to A and B isoforms (−11 and +11 isoforms) of the human, rat, and pig insulin receptors (HIR, RIR, and PIR) using standard methods. The relative affinities of these IRBPs for the various receptors is set forth in FIG. 2.

FIG. 2
Binding of IM peptides to IR from different species

| | HIR − | HIR + | RIR − | RIR + | PIR − | PIR + |
|---|---|---|---|---|---|---|
| | 11 | 11 | 11 | 11 | 11 | 11 |
| S519 | 26 | 31 | 3.2 | 3.8 | 0.5 | 1.4 |
| S557 | 25 | 29 | 3.5 | 5.1 | 1.5 | 2.3 |
| S597 | 46 | 61 | 17 | 25 | 2.9 | 7.5 |
| S636 | 106 | 117 | 50 | 79 | 32 | 68 |
| S667 | 111 | 127 | 44 | 112 | 47 | 84 |
| S733 | 281 | 290 | 179 | 211 | 111 | 228 |
| S671 | 86 | 98 | 39 | 65 | 23 | 58 |
| S660 | 52 | 58 | 6.5 | 9 | 5.5 | 12 |
| S696 | 3.3 | 3.1 | 0.3 | 0.2 | 0.3 | 0.8 |
| S574 | 8.3 | 8.2 | 0.9 | 2 | 0.2 | 0.4 |
| S700 | 70 | 53 | 10 | 13 | 1.4 | 4.3 |
| S626 | 59 | 78 | 11 | 12 | 1 | 3 |
| S726 | 83 | 121 | 13 | 7.7 | 1.3 | 5.9 |

| | | | |
|---|---|---|---|
| +11/−11 ratio | 1.1 | 1.4 | 2.5 |

As can be seen from FIG. 2, a number of IRBPs exhibit selectivity for the IR of a particular species, such as HIR, as opposed to RIR and/or PIR. Thus, this data illustrates an aspect of the invention that relates both to previously characterized IRBPs, disclosed in the prior patent documents, and the new IRBPs disclosed herein; namely that IRBPs can be selected based on their specificity for the IR of a particular species and that modifications to IRBAASs can modulate IR species specificity of such molecules. Thus, for example, the invention provides a method of specifically targeting HIR as opposed to PIR and/or RIR by selecting an IRBP specific therefor.

FIG. 2 also illustrates that IRBPs exhibit IR isoform specificity within a particular species (i.e., at least some IRBPs exhibit greater affinity for HIR+11 than HIR−11 and visa versa). This illustrates another novel aspect of the invention, that IRBPs can be used to specifically target particular IR isoforms and, thus, for example, can be used to treat IR-mediated conditions, disorders, etc. in particular target tissues based on such selections.

A number of other consequences arise from the species and isoform specificity of IRBPs, at least some of which are described elsewhere herein, which form additional aspects of the invention.

### Example 3

This Example demonstrates the ability of a novel IRBP of the invention to regulate glucose levels as compared to human insulin in a relevant animal model.

IRBP S667 was and human insulin were administered to wistar rats (S667 at 1.25 nmol/kg; 2.5 nmol/kg; and 5 nmol/kg; human insulin (HI) at 1.25 nmol/kg) using standard techniques, such as are described in the prior patent documents. Glucose measurements were taken 20 minutes prior to administration, at administration, and 20; 40; 60; 80; 120; and 180 minutes after administration. These measurements are compared in FIG. 3.

As can be seen from FIG. 3, S667, a novel IRBP, was able to lower blood glucose to levels similar to human insulin at similar dosages. The results in FIG. 3 also demonstrate that at least across some range of dosage, the IRBP exhibits dose-dependent IR-mediated glucose level modulation.

All references, including publications, patent applications, and patents, cited herein are hereby incorporated by reference to the same extent as if each reference were individually and specifically indicated to be incorporated by reference and were set forth in its entirety herein.

All headings and sub-headings are used herein for convenience only and should not be construed as limiting the invention in any way.

Any combination of the above-described elements in all possible variations thereof is encompassed by the invention unless otherwise indicated herein or otherwise clearly contradicted by context.

The use of the terms “a” and “an” and “the” and similar referents in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability, and/or enforceability of such patent documents.

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

Various formulas are used herein as convenient shorthand method of describing groups of amino acid sequences. It will be understood that such a formulaic description of groups of sequences provides literal support for each sequence falling within these formulas as an individual aspect of the invention, unless otherwise stated or clearly contradicted by context (numerous particular examples of sequences encompassed by such formulas are provided herein—see, e.g., Table 2).

This invention includes all modifications and equivalents of the subject matter recited in the aspects presented herein to the maximum extent permitted by applicable law.

1. An insulin receptor binding protein (IRBP) comprising:
(1) an N-terminal insulin-receptor binding amino acid sequence (IRBAAS) according to one or more of Formulas 6a-6g, wherein
(a) Formula 6a consists of the sequence Xaa₁ Leu Glu Xaa₄ Glu Trp Xaa₇ Xaa₈ Xaa₉ Xaa₁₀ Xaa₁₁ Xaa₁₂ Val Tyr Xaa₁₅ Xaa₁₆ Xaa₁₇ Xaa₁₈ (SEQ ID NO:6), wherein Xaa₁, Xaa₄, Xaa₇, Xaa₈, Xaa₉, Xaa₁₀, Xaa₁₂, Xaa₁₅, Xaa₁₆, and Xaa₁₇ are any suitable amino acid residues and Xaa₁₁, Xaa₁₈, or both are any suitable residue other than Cys;
(b) Formula 6b consists of Formula 6a, wherein Xaa₁₁ is an Ala or Glu, Xaa₁₈ is an Ala or Glu, or both Xaa₁ₗ and Xaa₁₈ are, independently, Ala or Glu residues;
(c) Formula 6c consists of the sequence Ser Leu Glu Glu Glu Tip Ala Gln Ile Glu Xaa₁₁ Glu Val Trp Gly Arg Gly Xaa₁₈ (SEQ ID NO:7), wherein Xaa₁₁ and/or Xaa₁₈ are any suitable residue other than Cys;
(d) Formula 6d consists of a sequence according to Formula 6c, wherein Xaa₁₁ and/or Xaa₁₈ is an Ala residue;
(e) Formula 6e consists of the sequence Xaa₁ Leu Glu Xaa₄ Glu Tip Xaa₇ Xaa₈ Xaa₉ Xaa₁₀ Xaa₁₁ Xaa₁₂ Val Tyr Xaa₁₅ Xaa₁₆ Xaa₁₇ Xaa₁₈ (SEQ ID NO:10), wherein (a) Xaa₁₁ and/or Xaa₁₈ are Cys residues or other suitable amino acid residues and (b) one or more of Xaa₄, Xaa₇, Xaa₈, Xaa₁₅, and Xaa₁₇ are independently degradation-resistant unusual amino acid residues and/or moieties;
(f) Formula 6f consists of the sequence Ser Leu Glu Glu Glu Tip Ala Gln Ile Xaa₁₀ Xaa₁₁ Glu Val Tip Gly Arg Gly Xaa₁₈ (SEQ ID NO:11), wherein Xaa₁₀ is Glu or Gln and Xaa₁₁ and Xaa₁₈ are any suitable residues; and
(g) Formula 6g consists of the sequence Xaa₁ Leu Glu Xaa₄ Glu Trp Xaa₇ Xaa₈ Xaa₉ Xaa₁₀ Xaa₁₁ Xaa₁₂ Val Tyr Xaa₁₅ Xaa₁₆ Xaa₁₇ Xaa₁₈ (SEQ ID NO:12), wherein Xaa₁, Xaa₄, Xaa₇, Xaa₈, Xaa₉, Xaa₁₀, Xaa₁₂, Xaa₁₅, Xaa₁₆, and Xaa₁₇ are any suitable amino acid residues; Xaa₁₈ is Cys or any other suitable residue; and Xaa₁₁ is Cys or any other suitable residue;
and
(ii) a C-terminal IRBAAS according to (a) Formula 1, (b) one or more of Formulas 1a-1g, (c) Formula 2, or (d) Formula 2a; wherein
(a) Formula 1 is Xaa₁ Tyr Xaa₃ Trp Xaa₅;
(b) Formula 1a consists of Formula 1 wherein (I) Xaa₁, Xaa₅, or both are either (A) degradation-resistant unusual amino acid residues or degradation-resistant chemical moieties or (B) Phe residues, and (II) Xaa₃ is a degradation-resistant unusual amino acid residue, a non-amino acid residue degradation resistant chemical moiety, or any suitable other amino acid residue;
(c) Formula 1b consists of the sequence Xaa₁ Tyr Xaa₃ Trp Xaa₅ Xaa₆ Xaa₇ Xaa₈ Xaa₉, wherein Xaa₆ is any suitable amino acid residue; Xaa₇ is any suitable residue; Xaa₈ is selected from Gln, Glu, Ala, and Lys; and Xaa₉ is a hydrophobic amino acid;
(d) Formula 1c consists of the sequence Xaa₁ Tyr Xaa₃ Trp Xaa₅ Glu Arg Gln Leu (SEQ ID NO: 1), wherein (I) Xaa₁, Xaa₅, or both are either (A) degradation-resistant unusual amino acid residues or degradation-resistant chemical moieties or (B) Phe residues, and (II) Xaa₃ is a degradation-resistant unusual amino acid residue, a non-amino acid residue degradation resistant chemical moiety, or any suitable other amino acid residue;
(e) Formula 1d consists of the sequence Xaa₁ Tyr Xaa₃ Trp Xaa₅ Glu Arg Gln Leu Gly (SEQ ID NO:2), wherein (I) Xaa₁, Xaa₅, or both are either (A) degradation-resistant unusual amino acid residues or degradation-resistant chemical moieties or (B) Phe residues, and (II) Xaa₃ is a degradation-resistant unusual amino acid residue, a non-amino acid residue degradation resistant chemical moiety, or any suitable other amino acid residue;
(f) Formula 1e consists of the sequence Xaa₁ Tyr Gly Tip Xaa₅ Glu Arg Gln Xaa₉ Gly (SEQ ID NO:3), wherein Xaa₁ and Xaa₅ are independently a Phe or a degradation-resistant amino acid residue or a non-amino acid residue degradation-resistant chemical moiety; and Xaa₉ is any suitable residue;
(g) Formula 1f consists of the sequence Xaa₁ Tyr Xaa₃ Tip Xaa₅ Glu Arg Gln Leu Gly (SEQ ID NO:4), wherein (I) Xaa₁, Xaa₅, or both are either (A) degradation-resistant unusual amino acid residues or degradation-resistant chemical moieties or (B) Phe residues, and (II) Xaa₃ is a Gly or His residue;
(h) Formula 1g consists of the sequence Xaa₁ Tyr Xaa₃ Trp Xaa₅ Xaa₆ Xaa₇ Xaa₈ Xaa₉ Xaa₁₀, wherein Xaa₁ is a Phe or degradation-resistant residue/moiety; Xaa₅ is a Phe or degradation-resistant moiety/residue; Xaa₃ is any suitable residue; Xaa₆-Xaa₈ are any suitable residues; Xaa₉ is any suitable residue or is missing; and Xaa₁₀ is a hydrophobic residue;
(i) Formula 2 consists of X₆X₇X₈X₉X₁₀X₁₁X₁₃, wherein X₆ and X₇ are aromatic amino acids; X₈, X₉, X₁₁ and X₁₂ are any amino acid; and X₁₀ and X₁₃ are hydrophobic amino acids; and
(j) Formula 2a consists of the sequence Ser Glu Gly Phe Tyr Asn Ala Ile Glu Leu Leu Ser (SEQ ID NO:5).

2. An IRBP as defined in claim 1, comprising a N-terminal IRBAAS according to Formula 6 and a C-terminal IRBAAS according to one or more of Formulas 1a-1g or Formula 2a.

3. The IRBP as defined in claim 1, wherein the IRBP has an affinity for HIR that is at least about 10% that of human insulin.

4. The IRBP as defined in claim 1, wherein the IRBP has an affinity for HIR that is greater than that of human insulin.

5. The IRBP of claim 1, wherein the IRBP is able to lower blood glucose levels at least about 50% as efficiently as insulin.

6. The IRBP of claim 1, wherein the IRBP consists essentially of a dimer of two different and respectively Site-1-binding and Site-2-binding IRBAASs, oriented Site-2 to Site-1 and linked C-N terminus.

7. The IRBP of claim 1, wherein the IRBP is about 10-60 amino acids in length.

8. The IRBP of claim 7 wherein the IRBP is about 25-50 amino acids in length.

9. The IRBP of claim 1, wherein the IRBP is selective for HIR−11 or HIR+11.

10. The IRBP of claim 1, wherein the IRBP exhibits at least about 50-fold greater resistance to digestive enzyme degradation than S597.

11. The IRBP of claim 10, wherein the IRBP exhibits at least about 100-fold greater resistance to digestive enzyme degradation than S597.

12. A pharmaceutically acceptable IRBP composition comprising a therapeutically effective amount of the IRBP of claim 1 and one or more pharmaceutically acceptable carriers.

13. A method of modulating a physiological activity associated with IR activity in a patient comprising delivering to the patient a physiological effective amount of an IRBP of claim 1.

14. The method of claim 13, wherein the IRBP is delivered by administering to the patient a nucleic acid comprising a sequence that codes for the IRBP and is expressible in the patient.

15. A method of treating diabetes in a patient comprising delivering the patient a therapeutically effective amount of the IRBP according to claim 1.

16. The method of claim 15, wherein the method further comprises administering to the patient a long-acting insulin analog, wherein the amount of IRBP and amount of long-acting insulin analog are together therapeutically effective.

17. The method of claim 15, wherein the method further comprises administering to the patient a non-insulin and non-insulin-analog anti-diabetic agent.

18. (canceled)

19. The method of claim 15, wherein the IRBP composition is administered to the patient by pulmonary delivery.

20. The method of claim 15, wherein the IRBP composition is administered to the patient by oral delivery.

21.-24. (canceled)
